(19) United States
(12) Patent Application Publication
(10) Pub. No.: US 2026/0015597 A1
(43) Pub. Date: Jan. 15, 2026

(54) COMPOSITIONS AND METHODS FOR CLEAVING VIRAL GENOMES

(71) Applicants: Alexandra Howell, Hanover, NH (US); Matthew S. Hayden, Lebanon, NH (US)

(72) Inventors: Alexandra Howell, Hanover, NH (US); Matthew S. Hayden, Lebanon, NH (US)

(21) Appl. No.: 18/039,905

(22) PCT Filed: Dec. 1, 2021
(86) PCT No.: PCT/US21/61398

§ 371 Date: Jun. 1, 2023

Related U.S. Application Data
(60) Provisional application No. 63/120,089, filed on Dec. 1, 2020.

(51) Int. Cl. C12N 9/22; A61K 38/46; C12N 15/113; C12N 15/90

(52) U.S. Cl. CPC C12N 9/226; A61K 38/465; C12N 15/1131; C12N 15/902; C12N 2310/20

(57) ABSTRACT

Disclosed are methods of inactivating a virus in a cell comprising administering to a cell comprising a viral genome, a Cas12d or Cas12e endonuclease, or a nucleic acid construct that encodes the Cas12d or Cas12e endonuclease; and a first guide RNA (gRNA), or a nucleic acid construct that encodes the first gRNA, wherein the Cas12d or Cas12e endonuclease cleaves the viral genome at a first target sequence and a second target sequence in the viral genome.

### CROSS-REFERENCE TO RELATED APPLICATIONS

This application claims the benefit of U.S. Provisional Patent Application No. 63/120,089 filed on Dec. 1, 2020, each of which is incorporated by reference herein in its entirety.

### STATEMENT REGARDING FEDERALLY SPONSORED RESEARCH

This invention was made with government support under 1R43GM133289-01 awarded by National Institute of General Medical Sciences. The government has certain rights in the invention.

### BACKGROUND

CRISPR/Cas techniques have been applied to a viral system by transfecting human cell lines with plasmids expressing the guide RNA and Cas9 genes that cleaved a single region in a model proviral sequence carried on a plasmid. Using similar techniques, it was demonstrated that use of multiple guide RNAs (gRNAs) targeted to both the 5′ and 3′ ends of the provirus could excise the entire proviral genome in cell lines or inactivate the virus through the introduction of mutations. The Cas9 and gRNAs used in these systems result in blunt end cleavage at both ends that are acted upon by error prone DNA repair mechanisms resulting in small mutations, deletions or insertions that disrupt the gRNA target sequence.

Use of the prior techniques left behind challenges. Specifically, challenges remain in the methods of achieving removal of the viral or pro-viral genome and also in delivery of guide RNA and Cas9 genes, especially to resting leukocytes either in vivo or ex vivo as a therapeutic modality for treating virus-infected patients. What is needed are techniques that overcome the challenges of these prior techniques.

### BRIEF SUMMARY

Disclosed are methods of inactivating a virus in a cell comprising administering to a cell comprising a viral genome a Cas12d or Cas12e endonuclease, or a nucleic acid construct that encodes the Cas12d or Cas12e endonuclease; and a first guide RNA (gRNA), or a nucleic acid construct that encodes the first gRNA, wherein the Cas12d or Cas12e endonuclease cleaves the viral genome at a first target sequence and a second target sequence in the viral genome.

Disclosed are methods of inactivating a virus in a cell comprising administering to a cell comprising a viral genome a Cas12d or Cas12e endonuclease, or a nucleic acid construct that encodes the Cas12d or Cas12e endonuclease; a first gRNA, or a nucleic acid construct that encode the first gRNA, wherein the first gRNA is complementary to a first target sequence in the viral genome of the virus; and a second gRNA, or a nucleic acid construct that encodes the second gRNA, wherein the second gRNA is complementary to a second target sequence in the viral genome of the virus; wherein the first gRNA hybridizes to the first target sequence in the viral genome and the second gRNA hybridizes to the second target sequence in the viral genome resulting in Cas12d or Cas12e endonuclease cleavage at the first target sequence and the second target sequence in the viral genome.

Additional advantages of the disclosed method and compositions will be set forth in part in the description which follows, and in part will be understood from the description, or may be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate several embodiments of the disclosed method and compositions and together with the description, serve to explain the principles of the disclosed method and compositions.

FIG. 1 shows the highly efficient targeted excision of HBV in a human cell line.

FIG. 2 shows cutting of portions of the HBV genome in vitro using single sgRNAs directed against HBV and CasX2.

FIG. 3 shows an in vitro analysis of off-target cleavage events with SpCas9/sgRNA and CasX2/sgRNA targeting HBV. SEQ ID NO:173 is the top sequence. SEQ ID NO:174 is the bottom sequence.

FIG. 4 shows a disruption of HBV HBx gene in human cell lines using CasX1 and HBx sgRNAT. PCR amplified.region of the HBx gene was treated with or without T7 endonuclease to detect the presence of mutations or INDELs. SEQ ID NO: 175 is the top sequence and SEQ ID NO:176 is the bottom sequence.

FIG. 5 shows excision of portions of the HBV genome in human cell lines using CasX with pairs of matched gRNAs.

FIG. 6 shows Sanger sequencing results demonstrating targeted excision of HBV genomic DNA. sgRNA target sites, followed by the sequence obtained upon sanger sequencing of individual sequences obtained after PCR of isolated DNA from cells transfected with plasmid DNA containing the HBV genome and encoding CasX and sgRNAs indicated. For the PreS gene the top sequence is SEQ ID NO:177 and the bottom sequence is SEQ ID NO:178. For the X gene the top sequence is SEQ ID NO:179 and the bottom sequence is SEQ ID NO: 180. From top to bottom the remaining sequences are SEQ ID NO:181, SEQ ID NO:182, SEQ ID NO:183, SEQ ID NO:184, SEQ ID NO:185, SEQ ID NO: 186, SEQ ID NO: 187, SEQ ID NO:188, SEQ ID NO:189, SEQ ID NO:190, SEQ ID NO:191, and SEQ ID NO: 192.

FIG. 7 is a T7 assay using specific gRNAs.

FIG. 8 depicts a quantitative PCR that is performed prior to the T7 endonuclease assay.

FIG. 9 shows a co-transfection experiment with new gRNAs.

FIGS. 10A and 10B show a restriction map. A) shows the fragments that are amplified from each of the parent vectors. B) shows the final vector after the two fragments from A) come together.

FIG. 11 shows a cloning strategy.

FIG. 12 shows a cloning a strategy.

FIG. 13 shows the gRNAs used in the experiments herein. Sequences top to bottom are SEQ ID NO:193, SEQ ID NO:194, SEQ ID NO:195, SEQ ID NO:196, SEQ ID NO:197, SEQ ID NO:198, SEQ ID NO:199, SEQ ID NO:200, SEQ ID NO:201, SEQ ID NO:202, SEQ ID NO:203, SEQ ID NO:204, SEQ ID NO:205, SEQ ID NO:206, SEQ ID NO:207, SEQ ID NO:208, SEQ ID NO:209.

FIG. 14 shows the gRNAs used in the experiments of FIG. 2. Sequences top to bottom are SEQ ID NO:210, SEQ ID NO:211, SEQ ID NO:212, SEQ ID NO:213, SEQ ID NO:214, SEQ ID NO:215, SEQ ID NO:216, SEQ ID NO:217, SEQ ID NO:218, SEQ ID NO:219, SEQ ID NO:220, SEQ ID NO:221, SEQ ID NO:222, SEQ ID NO:223, SEQ ID NO:224.

FIG. 15 shows an example sequence of CasX1 and CasX2.

FIG. 16 shows an example sequence of CasY15.

FIG. 17 shows an example construct carrying CasX2.

FIGS. 18A-D show examples of HIV-1 (A), HBV (B), HTLV-1 (C) and JCV (D) Cas12d gRNA pairs. Depicted are gRNA target sites (protospacer) and adjacent or overlapping regions of microhomology (MH) shared between the two gRNAs shown for each virus. A) top left SEQ ID NO:228; bottom left SEQ ID NO:229; top right SEQ ID NO:230, bottom right SEQ ID NO:231; B) top left SEQ ID NO:232; bottom left SEQ ID NO:233; top right SEQ ID NO:234, bottom right SEQ ID NO:235; C) top left SEQ ID NO:236; bottom left SEQ ID NO:237; top right SEQ ID NO:238, bottom right SEQ ID NO:239; D) top left SEQ ID NO:240; bottom left SEQ ID NO:241; top right SEQ ID NO:242, bottom right SEQ ID NO: 243.

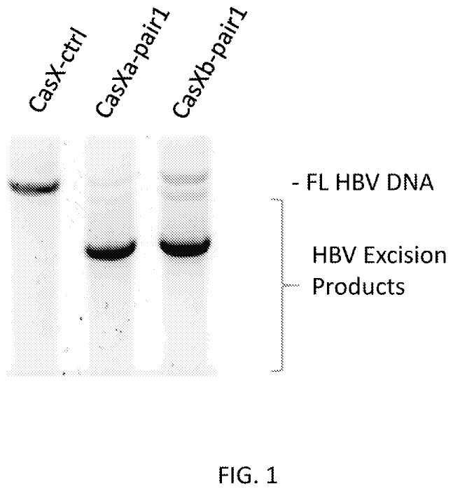

### DETAILED DESCRIPTION

The disclosed method and compositions may be understood more readily by reference to the following detailed description of particular embodiments and the Example included therein and to the Figures and their previous and following description.

It is to be understood that the disclosed method and compositions are not limited to specific synthetic methods, specific analytical techniques, or to particular reagents unless otherwise specified, and, as such, may vary. It is also to be understood that the terminology used herein is for the purpose of describing particular embodiments only and is not intended to be limiting.

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. Thus, if a class of molecules A, B, and C are disclosed as well as a class of molecules D, E, and F and an example of a combination molecule, A-D is disclosed, then even if each is not individually recited, each is individually and collectively contemplated. Thus, in this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

### A. Definitions

It is understood that the disclosed method and compositions are not limited to the particular methodology, protocols, and reagents described as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a”, “an”, and “the” include plural reference unless the context clearly dictates otherwise. Thus, for example, reference to “a gRNA” includes a plurality of such gRNAs, reference to “the target sequence” is a reference to one or more target sequences and equivalents thereof known to those skilled in the art, and so forth.

As used herein, the term “subject” refers to the target of administration, e.g., a human. Thus the subject of the disclosed methods can be a vertebrate, such as a mammal, a fish, a bird, a reptile, or an amphibian. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.). In one aspect, a subject is a mammal. In another aspect, a subject is a human. The term does not denote a particular age or sex. Thus, adult, child, adolescent and newborn subjects, as well as fetuses, whether male or female, are intended to be covered.

As used herein, the terms “treatment,” “treating,” and the like, refer to obtaining a desired pharmacologic and/or physiologic effect. In some aspects, “treat” is meant to mean administer a gRNA, endonuclease or composition described herein to a subject, such as a human or other mammal (for example, an animal model), that has a disease or condition, in order to prevent or delay a worsening of the effects of the disease or condition, or to partially or fully reverse the effects of the disease or condition. In some aspects, the disease or condition can be a viral infection. Treatment may be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition. In some embodiments, treatment comprises delivery of one or more of the disclosed gRNAs, endonucleases or compositions to a subject.

As used herein, “prevent” is meant to mean minimize the chance that a subject who has an increased susceptibility for developing disease, disorder or condition will develop the disease, disorder or condition. For example, prevent as used herein can mean minimize the chance that a subject who has an increased susceptibility for developing a viral infection will become infected.

As used herein, the terms “administering” and “administration” refer to any method of providing a disclosed polypeptide, polynucleotide, vector, composition, or a pharmaceutical preparation to a subject. Such methods are well known to those skilled in the art and include, but are not limited to: oral administration, transdermal administration, administration by inhalation, nasal administration, topical administration, intravaginal administration, ophthalmic administration, intraaural administration, intracerebral administration, rectal administration, sublingual administration, buccal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition. In an aspect, the skilled person can determine an efficacious dose, an efficacious schedule, or an efficacious route of administration for a disclosed composition or a disclosed conjugate so as to treat a subject or induce apoptosis. In an aspect, the skilled person can also alter or modify an aspect of an administering step so as to improve efficacy of a disclosed polypeptide, polynucleotide, vector, composition, or a pharmaceutical preparation.

The terms “polynucleotide” and “nucleic acid,” used interchangeably herein, refer to a polymeric form of nucleotides of any length, either ribonucleotides or deoxynucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases. The terms “polynucleotide” and “nucleic acid” should be understood to include, as applicable to the embodiment being described, single-stranded (such as sense or antisense) and double-stranded polynucleotides.

The terms “polypeptide,” “peptide,” and “protein”, are used interchangeably herein, refer to a polymeric form of amino acids of any length, which can include genetically coded and non-genetically coded amino acids, chemically or biochemically modified or derivatized amino acids, and polypeptides having modified peptide backbones. The term includes fusion proteins, including, but not limited to, fusion proteins with a heterologous amino acid sequence, fusions with heterologous and homologous leader sequences, with or without N-terminal methionine residues; immunologically tagged proteins; and the like.

The term “naturally-occurring” as used herein as applied to a nucleic acid, a protein, a cell, or an organism, refers to a nucleic acid, cell, protein, or organism that is found in nature. In some aspects, the term “naturally-occurring” can mean “wild-type.”

“Recombinant,” as used herein, means that a particular nucleic acid (DNA or RNA) is the product of various combinations of cloning, restriction, and/or ligation steps resulting in a construct having a structural coding or non-coding sequence distinguishable from endogenous nucleic acids found in natural systems. Generally, DNA sequences encoding the structural coding sequence can be assembled from cDNA fragments and short oligonucleotide linkers, or from a series of synthetic oligonucleotides, to provide a synthetic nucleic acid which is capable of being expressed from a recombinant transcriptional unit contained in a cell or in a cell-free transcription and translation system. Such sequences can be provided in the form of an open reading frame uninterrupted by internal non-translated sequences, or introns, which are typically present in eukaryotic genes. Genomic DNA comprising the relevant sequences can also be used in the formation of a recombinant gene or transcriptional unit. Sequences of non-translated DNA may be present 5′ or 3′ from the open reading frame, where such sequences do not interfere with manipulation or expression of the coding regions, and may indeed act to modulate production of a desired product by various mechanisms (see “DNA regulatory sequences”, below).

Thus, e.g., the term “recombinant” polynucleotide or “recombinant” nucleic acid refers to one which is not naturally occurring, e.g., is made by the artificial combination of two otherwise separated segments of sequence through human intervention. This artificial combination is often accomplished by either chemical synthesis means, or by the artificial manipulation of isolated segments of nucleic acids, e.g., by genetic engineering techniques. Such is usually done to replace a codon with a redundant codon encoding the same or a conservative amino acid, while typically introducing or removing a sequence recognition site. Alternatively, it is performed to join together nucleic acid segments of desired functions to generate a desired combination of functions. This artificial combination is often accomplished by either chemical synthesis means, or by the artificial manipulation of isolated segments of nucleic acids, e.g., by genetic engineering techniques.

Similarly, the term “recombinant” polypeptide refers to a polypeptide which is not naturally occurring, e.g., is made by the artificial combination of two otherwise separated segments of amino sequence through human intervention. Thus, e.g., a polypeptide that comprises a heterologous amino acid sequence is recombinant.

By “construct” or “vector” is meant a recombinant nucleic acid, generally recombinant DNA, which has been generated for the purpose of the expression and/or propagation of a specific nucleotide sequence(s), or is to be used in the construction of other recombinant nucleotide sequences.

A “host cell,” as used herein, denotes an in vivo or in vitro eukaryotic cell, a prokaryotic cell, or a cell from a multicellular organism (e.g., a cell line) cultured as a unicellular entity, which eukaryotic or prokaryotic cells can be, or have been, used as recipients for a nucleic acid (e.g., an expression vector), and include the progeny of the original cell which has been genetically modified by the nucleic acid. It is understood that the progeny of a single cell may not necessarily be completely identical in morphology or in genomic or total DNA complement as the original parent, due to natural, accidental, or deliberate mutation. A “recombinant host cell” (also referred to as a “genetically modified host cell”) is a host cell into which has been introduced a heterologous nucleic acid, e.g., an expression vector. For example, a subject prokaryotic host cell is a genetically modified prokaryotic host cell (e.g., a bacterium), by virtue of introduction into a suitable prokaryotic host cell of a heterologous nucleic acid, e.g., an exogenous nucleic acid that is foreign to (not normally found in nature in) the prokaryotic host cell, or a recombinant nucleic acid that is not normally found in the prokaryotic host cell; and a subject eukaryotic host cell is a genetically modified eukaryotic host cell, by virtue of introduction into a suitable eukaryotic host cell of a heterologous nucleic acid, e.g., an exogenous nucleic acid that is foreign to the eukaryotic host cell, or a recombinant nucleic acid that is not normally found in the eukaryotic host cell.

A polynucleotide or polypeptide has a certain percent “sequence identity” to another polynucleotide or polypeptide, meaning that, when aligned, that percentage of bases or amino acids are the same, and in the same relative position, when comparing the two sequences. Sequence similarity can be determined in a number of different manners. To determine sequence identity, sequences can be aligned using the methods and computer programs, including BLAST, available over the world wide web at ncbi.nlm.nih.gov/BLAST. See, e.g., Altschul et al. (1990), J. Mol. Biol. 215:403-10. Another alignment algorithm is FASTA, available in the Genetics Computing Group (GCG) package, from Madison, Wis., USA, a wholly owned subsidiary of Oxford Molecular Group, Inc. Other techniques for alignment are described in Methods in Enzymology, vol. 266: Computer Methods for Macromolecular Sequence Analysis (1996), ed. Doolittle, Academic Press, Inc., a division of Harcourt Brace & Co., San Diego, Calif, USA. Of particular interest are alignment programs that permit gaps in the sequence. The Smith-Waterman is one type of algorithm that permits gaps in sequence alignments. See Meth. Mol. Biol. 70: 173-187 (1997). Also, the GAP program using the Needleman and Wunsch alignment method can be utilized to align sequences. See J. Mol. Biol. 48: 443-453 (1970).

As used herein, “hybridization” means the pairing of an oligonucleotide with a complementary nucleic acid sequence. Such pairing typically involves hydrogen bonding, which may be Watson-Crick, Hoogsteen or reversed Hoogsteen hydrogen bonding, between complementary nucleoside or nucleotide bases (nucleobases) of an oligonucleotide (e.g. gRNA) and a target nucleic acid sequence (e.g., wherein the oligonucleotide comprises the reverse complementary nucleotide sequence of the corresponding region of the target nucleic acid). In particular embodiments, an oligonucleotide specifically hybridizes to a target nucleic acid. The terms “specifically hybridizes” and “specifically hybridizable” are used interchangeably herein to indicate a sufficient degree of complementarity such that stable and specific binding occurs between the oligonucleotide and the target nucleic acid (i.e., DNA or RNA). It is understood that an oligonucleotide need not be 100% complementary to its target nucleic acid sequence to be specifically hybridizable. In particular embodiments, an oligonucleotide is considered to be specifically hybridizable when binding of the oligonucleotide to a target nucleic acid sequence interferes with the normal function of the target nucleic acid and results in a loss or altered utility or expression therefrom. In preferred embodiments, there is a sufficient degree of complementarity between the oligonucleotide and target nucleic acid to avoid or minimize non-specific binding of the oligonucleotide to undesired non-target sequences under the conditions in which specific binding is desired (e.g., under physiological conditions in the case of in vivo assays or therapeutic treatment, and in the case of in vitro assays, under conditions in which the assays are performed). It is well within the level of skill of scientists in the oligonucleotide field to routinely determine when conditions are optimal for specific hybridization to a target nucleic acid with minimal non-specific hybridization events. Thus, in some embodiments, oligonucleotides in the complexes of the invention include 1, 2, or 3 base substitutions compared to the corresponding complementary sequence of a region of a target DNA or RNA sequence to which it specifically hybridizes. In some embodiments, the location of a non-complementary nucleobase is at the 5′ end or 3′ end of an antisense oligonucleotide. In additional embodiments, a non-complementary nucleobase is located at an internal position in the oligonucleotide. When two or more non-complementary nucleobases are present in an oligonucleotide, they may be contiguous (i.e., linked), non-contiguous, or both. In some embodiments, the oligonucleotides in the complexes of the invention have at least 85%, at least 90%, or at least 95% sequence identity to a target region within the target nucleic acid. In other embodiments, oligonucleotides have 100% sequence identity to a polynucleotide sequence within a target nucleic acid. Percent identity is calculated according to the number of bases that are identical to the corresponding nucleic acid sequence to which the oligonucleotide being compared. This identity may be over the entire length of the oligomeric compound (i.e., oligonucleotide), or in a portion of the oligonucleotide (e.g., nucleobases 1-20 of a 27-mer may be compared to a 20-mer to determine percent identity of the oligonucleotide to the oligonucleotide). Percent identity between an oligonucleotide and a target nucleic acid can routinely be determined using alignment programs and BLAST programs (basic local alignment search tools) known in the art (see, e.g., Altschul et al., J. Mol. Biol., 1990, 215, 403-410; Zhang and Madden, Genome Res., 1997, 7, 649-656).

“Optional” or “optionally” means that the subsequently described event, circumstance, or material may or may not occur or be present, and that the description includes instances where the event, circumstance, or material occurs or is present and instances where it does not occur or is not present.

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinence of the cited documents. It will be clearly understood that, although a number of publications are referred to herein, such reference does not constitute an admission that any of these documents forms part of the common general knowledge in the art.

Throughout the description and claims of this specification, the word “comprise” and variations of the word, such as “comprising” and “comprises,” means “including but not limited to,” and is not intended to exclude, for example, other additives, components, integers or steps. In particular, in methods stated as comprising one or more steps or operations it is specifically contemplated that each step comprises what is listed (unless that step includes a limiting term such as “consisting of”), meaning that each step is not intended to exclude, for example, other additives, components, integers or steps that are not listed in the step.

As used herein, “CasX” issued interchangeably with the Cas12e, family of RNA-guided endonucleases and visa versa.

As used herein, “CasY” is used interchangeably with the Cas12d, family of RNA-guided endonucleases and visa versa.

### B. Methods

Disclosed are methods of inactivating a virus in a cell comprising administering to a cell comprising a viral genome, a Cas12d or Cas12e endonuclease, or a nucleic acid construct that encodes the Cas12d or Cas12e endonuclease; and a first guide RNA (gRNA), or a nucleic acid construct that encodes the first gRNA, wherein the Cas12d or Cas12e endonuclease cleaves the viral genome at a first target sequence and a second target sequence in the viral genome. In some aspects, the Cas12d or Cas12e endonuclease, or a nucleic acid construct that encodes the Cas12d or Cas12e endonuclease are comprised in a composition. Thus, in some aspects, a composition comprising a Cas12d or Cas12e endonuclease, or a nucleic acid construct that encodes the Cas12d or Cas12e endonuclease, can be administered to a cell.

Disclosed are methods of inactivating a virus in a cell comprising administering to a cell comprising a viral genome, a Cas12d or Cas12e endonuclease, or a nucleic acid construct that encodes the Cas12d or Cas12e endonuclease; a first guide RNA (gRNA), or a nucleic acid construct that encode the first gRNA, wherein the first gRNA is complementary to a first target sequence in the viral genome of the virus; and a second gRNA, or a nucleic acid construct that encodes the second gRNA, wherein the second gRNA is complementary to a second target sequence in the viral genome of the virus; wherein the first gRNA hybridizes to the first target sequence in the viral genome and the second gRNA hybridizes to the second target sequence in the viral genome resulting in Cas12e or Cas12d endonuclease cleavage at the first target sequence and the second target sequence in the viral genome.

In some aspects, the disclosed methods of inactivating a virus in a cell comprise a Cas12d or Cas12e endonuclease working with at least a first gRNA, but often a first and second gRNA, to cleave a viral genome at a first and second target sequence within the viral genome. The cleavage of the viral genome at a first and second target sequence results in inactivation of the virus. In some aspects, a portion of one or more viral genes, the entirety of one or more viral genes, or the entire viral genome can be removed due to the endonuclease cleavage, wherein the removal results in inactivation of the virus. In some aspects, removal of a portion of one or more viral genes, the entirety of one or more viral genes, or the entire viral genome refers to removing it from the cellular genome.

In some aspects, the cell can be referred to as a host cell. In some aspects, the cell is eukaryotic. For example, the cell can be a human cell. In some aspects, the cell comprises both the cellular genome and the viral genome.

In some aspects, administering to a cell comprises administering to a subject comprising a cell. For example, administering a Cas12d or Cas12e endonuclease, or a nucleic acid construct that encodes the Cas12d or Cas12e endonuclease can comprise, but is not limited to, administering Cas12d or Cas12e endonuclease, or a nucleic acid construct that encodes the Cas12d or Cas12e endonuclease to a subject via intravenous, intramuscular, intracranial or subcutaneous injection. Once inside the subject, the Cas12d or Cas12e endonuclease, or a nucleic acid construct that encodes the Cas12d or Cas12e endonuclease, can enter a cell.

Disclosed are methods of treating a subject having a cell comprising a viral genome comprising administering to the subject a Cas12d or Cas12e endonuclease, or a nucleic acid construct that encodes the Cas12d or Cas12e endonuclease; a first gRNA, or a nucleic acid construct that encode the first gRNA, wherein the first gRNA is complementary to a first target sequence in the viral genome of the virus; and a second gRNA, or a nucleic acid construct that encodes the second gRNA, wherein the second gRNA is complementary to a second target sequence in the viral genome of the virus; wherein the first gRNA hybridizes to the first target sequence in the viral genome and the second gRNA hybridizes to the second target sequence in the viral genome resulting in Cas12e or Cas12d endonuclease cleavage at the first target sequence and the second target sequence in the viral genome. In some aspects, a subject having a cell comprising a viral genome is a subject having a viral infection. Thus, disclosed are methods of treating a viral infection. In some aspects, treating a subject can refer to removing the viral genome from the subject's cellular genome so that the virus cannot continue to replicate, infect, or cause disease symptoms.

### 1. Guide RNAs

Disclosed herein are gRNAs that can be used in the methods described herein. Examples of gRNAs include, but are not limited to the gRNAs disclosed in the Figures, Table 2 or in the Examples provided herein. Disclosed herein are gRNAs that specifically bind to a target sequence, including, but not limited to the target sequences of Table 1 or in the Figures or Examples provided herein.

In some aspects, a gRNA is a nucleic acid molecule that binds to a Cas12d or Cas12e endonuclease, forming a ribonucleoprotein complex (RNP), and targets the complex to a specific location within a target nucleic acid (e.g., a target sequence). It is to be understood that in some cases, a hybrid DNA/RNA can be made such that a gRNA includes DNA bases in addition to RNA bases, but the term “gRNA” is still used to encompass such a molecule herein.

As described herein, a gRNA can include two segments, a targeting segment (CRISPR RNA (crRNA)) and a protein-binding segment (transactivating crRNA (tracrRNA)). The targeting segment of a gRNA includes a nucleotide sequence (a guide sequence) that is complementary to (and therefore hybridizes with) a specific sequence (a target sequence) within a target nucleic acid (e.g., a viral genome). The protein-binding segment (or “protein-binding sequence”) interacts with (binds to) a Cas12d or Cas12e endonuclease. The protein-binding segment of a gRNA includes two complementary stretches of nucleotides that hybridize to one another to form a double stranded RNA duplex (dsRNA duplex), or stem loop. Site-specific binding and/or cleavage of a target nucleic acid (e.g., viral DNA) can occur at locations (e.g., target sequence of a target locus) determined by base-pairing complementarity between the gRNA (the guide sequence of the gRNA) and the target sequence of the target nucleic acid.

A gRNA and a Cas12d or Cas12e endonuclease form a complex (e.g., bind via non-covalent interactions). The gRNA provides target specificity to the complex by including a targeting segment, which includes a guide sequence (a nucleotide sequence that is complementary to a target sequence of a target nucleic acid). The Cas12d or Cas12e endonuclease of the complex provides the site-specific activity (e.g., cleavage activity provided by the Cas12d or Cas12e endonuclease). In other words, the Cas12d or Cas12e endonuclease is guided to a target nucleic acid sequence (e.g. a target sequence) by virtue of its association with the gRNA.

In some aspects, a gRNA can be a single guide RNA (sgRNA) that comprises both the crRNA and the tracrRNA. In some aspects, a gRNA can be formed after a crRNA and a tracrRNA hybridize (e.g. they have complementary segments) thus allowing the targeting sequence of the crRNA to bind to the target sequence while the protein binding segment of the tracrRNA brings the endonuclease which can then cleave the target sequence.

The targeting segment of a gRNA includes a guide sequence (i.e., a targeting sequence), which is a nucleotide sequence that is complementary to a sequence (a target sequence) in a target nucleic acid. In other words, the targeting segment of a gRNA can interact with a target nucleic acid (e.g., viral genome) in a sequence-specific manner via hybridization (i.e., base pairing). The guide sequence of a gRNA can be modified (e.g., by genetic engineering)/designed to hybridize to any desired target sequence (e.g., while taking the PAM into account, e.g., when targeting a dsDNA target) within a target nucleic acid (e.g., viral genome).

In some embodiments, the percent complementarity between the guide sequence and the target sequence of the target nucleic acid is 60% or more (e.g., 65% or more, 70% or more, 75% or more, 80% or more, 85% or more, 90% or more, 95% or more, 97% or more, 98% or more, 99% or more, or 100%). In some cases, the percent complementarity between the guide sequence and the target sequence of the target nucleic acid is 80% or more (e.g., 85% or more, 90% or more, 95% or more, 97% or more, 98% or more, 99% or more, or 100%). In some cases, the percent complementarity between the guide sequence and the target sequence of the target nucleic acid is 90% or more (e.g., 95% or more, 97% or more, 98% or more, 99% or more, or 100%). In some cases, the percent complementarity between the guide sequence and the target sequence of the target nucleic acid is 100%.

In some cases, the percent complementarity between the guide sequence and the target sequence of the target nucleic acid is 60% or more (e.g., 70% or more, 75% or more, 80% or more, 85% or more, 90% or more, 95% or more, 97% or more, 98% or more, 99% or more, or 100%) over 16 or more (e.g., 20 or more, 21 or more, 22 or more) contiguous nucleotides. In some cases, the percent complementarity between the guide sequence and the target sequence of the target nucleic acid is 80% or more (e.g., 85% or more, 90% or more, 95% or more, 97% or more, 98% or more, 99% or more, or 100%) over 16 or more (e.g., 20 or more, 21 or more, 22 or more) contiguous nucleotides. In some cases, the percent complementarity between the guide sequence and the target sequence of the target nucleic acid is 90% or more (e.g., 95% or more, 97% or more, 98% or more, 99% or more, or 100%) over 19 or more (e.g., 20 or more, 21 or more, 22 or more) contiguous nucleotides. In some cases, the percent complementarity between the guide sequence and the target sequence of the target nucleic acid is 100% over 19 or more (e.g., 20 or more, 21 or more, 22 or more) contiguous nucleotides.

In some cases, the percent complementarity between the guide sequence and the target sequence of the target nucleic acid is 60% or more (e.g., 70% or more, 75% or more, 80% or more, 85% or more, 90% or more, 95% or more, 97% or more, 98% or more, 99% or more, or 100%) over 169-25 contiguous nucleotides. In some cases, the percent complementarity between the guide sequence and the target sequence of the target nucleic acid is 80% or more (e.g., 85% or more, 90% or more, 95% or more, 97% or more, 98% or more, 99% or more, or 100%) over 19-25 contiguous nucleotides. In some cases, the percent complementarity between the guide sequence and the target sequence of the target nucleic acid is 90% or more (e.g., 95% or more, 97% or more, 98% or more, 99% or more, or 100%) over 16-25 contiguous nucleotides. In some cases, the percent complementarity between the guide sequence and the target sequence of the target nucleic acid is 100% over 16-25 contiguous nucleotides.

In some cases, the guide sequence has a length in a range of from 19-30 nucleotides (nt) (e.g., from 16-25, 16-22, 16-20, 20-30, 20-25, or 20-22 nt). In some cases, the guide sequence has a length in a range of from 19-25 nucleotides (nt) (e.g., from 16-22, 16-20, 20-25, 20-25, or 20-22 nt). In some cases, the guide sequence has a length of 16 or more nt (e.g., 16 or more, 17 or more, 18 or more, 19 or more, 20 or more, 21 or more, or 22 or more nt; 16 nt, 17 nt, 18 nt, 19 nt, 20 nt, 21 nt, 22 nt, 23 nt, 24 nt, 25 nt, etc.). In some cases the guide sequence has a length of 16 nt. In some cases the guide sequence has a length of 17 nt. In some cases the guide sequence has a length of 18 nt. In some cases the guide sequence has a length of 19 nt. In some cases the guide sequence has a length of 20 nt. In some cases the guide sequence has a length of 21 nt. In some cases the guide sequence has a length of 22 nt. In some cases the guide sequence has a length of 23 nt.

Examples of various Cas9 guide RNAs can be found in the art, and in some cases variations similar to those introduced into Cas9 guide RNAs can also be introduced into Cas12d or Cas12e gRNAs of the present disclosure. For example, see Jinek et al., Science. 2012 Aug. 17; 337(6096):816-21; Chylinski et al., RNA Biol. 2013 May; 10(5):726-37; Ma et al., Biomed Res Int. 2013; 2013:270805; Hou et al., Proc Natl Acad Sci USA. 2013 Sep. 24; 110(39):15644-9; Jinek et al., Elife. 2013; 2:e00471; Pattanayak et al., Nat Biotechnol. 2013 September; 31(9):839-43; Qi et al, Cell. 2013 Feb. 28; 152(5):1173-83; Wang et al., Cell. 2013 May 9; 153(4):910-8; Auer et. al., Genome Res. 2013 Oct. 31; Chen et. al., Nucleic Acids Res. 2013 Nov. 1; 41(20):e19; Cheng et. al., Cell Res. 2013 October; 23(10):1163-71; Cho et. al., Genetics. 2013 November; 195(3):1177-80; DiCarlo et al., Nucleic Acids Res. 2013 April; 41(7):4336-43; Dickinson et. al., Nat Methods. 2013 October; 10(10):1028-34; Ebina et. al., Sci Rep. 2013; 3:2510; Fujii et. al, Nucleic Acids Res. 2013 Nov. 1; 41(20):e187; Hu et. al., Cell Res. 2013 November; 23(11):1322-5; Jiang et. al., Nucleic Acids Res. 2013 Nov. 1; 41(20):e188; Larson et. al., Nat Protoc. 2013 November; 8(11):2180-96; Mali et. at., Nat Methods. 2013 October; 10(10):957-63; Nakayama et. al., Genesis. 2013 December; 51(12):835-43; Ran et. al., Nat Protoc. 2013 November; 8(11):2281-308; Ran et. al., Cell. 2013 Sep. 12; 154(6):1380-9; Upadhyay et. al., G3 (Bethesda). 2013 Dec. 9; 3(12):2233-8; Walsh et. al., Proc Natl Acad Sci USA. 2013 Sep. 24; 110(39):15514-5; Xie et. al., Mol Plant. 2013 Oct. 9; Yang et. al., Cell. 2013 Sep. 12; 154(6):1370-9; Briner et al., Mol Cell. 2014 Oct. 23; 56(2):333-9; and U.S. patents and patent applications: U.S. Pat. Nos. 8,906,616; 8,895,308; 8,889,418; 8,889,356; 8,871,445; 8,865,406; 8,795,965; 8,771,945; 8,697,359; 20140068797; 20140170753; 20140179006; 20140179770; 20140186843; 20140186919; 20140186958; 20140189896; 20140227787; 20140234972; 20140242664; 20140242699; 20140242700; 20140242702; 20140248702; 20140256046; 20140273037; 20140273226; 20140273230; 20140273231; 20140273232; 20140273233; 20140273234; 20140273235; 20140287938; 20140295556; 20140295557; 20140298547; 20140304853; 20140309487; 20140310828; 20140310830; 20140315985; 20140335063; 20140335620; 20140342456; 20140342457; 20140342458; 20140349400; 20140349405; 20140356867; 20140356956; 20140356958; 20140356959; 20140357523; 20140357530; 20140364333; and 20140377868; all of which are hereby incorporated by reference in their entirety.

In some aspects, the first gRNA is complementary to the first target sequence and the second target sequence in the viral genome. In some aspects, a single gRNA can be complementary to a first target sequence and a second target sequence in a viral genome when the viral genome has repeated sequences. For example, this can happen with retroviruses having long terminal repeats (LTRs) at each end (5′ and 3′) of the viral genome wherein the LTR is the same at the 5′ end of the viral genome and the 3′ end of the viral genome. Therefore, a gRNA, such as a first gRNA, can be complementary to a single sequence that is present at both the 5′ end of the viral genome and 3′ end of viral genome. For example, a first target sequence and a second target sequence can be a single sequence within the LTR. A first target sequence can be present in the 5′ LTR while the second target sequence can be present in the 3′ LTR

In some aspects, the first gRNA hybridizes to the first target sequence and the second target sequence in the viral genome resulting in Cas12d or Cas12e endonuclease cleavage at the first target sequence and the second target sequence in the viral genome.

Thus, in some aspects, a single gRNA can be used to cleave a viral genome in two different locations. Also disclosed is the use of at least two gRNAs to cleave a viral genome in two different locations.

In some aspects, the disclosed methods can further comprise a second gRNA. In some aspects, the first gRNA is complementary to the first target sequence of the viral genome and the second gRNA is complementary to the second target sequence in the viral genome of the virus. In some aspects, the first gRNA hybridizes to the first target sequence in the viral genome and the second gRNA hybridizes to the second target sequence in the viral genome resulting in Cas12d or Cas12e endonuclease cleavage at the first target sequence and the second target sequence in the viral genome.

In some aspects, the nucleic acid construct that encodes the first gRNA and the nucleic acid construct that encodes the second gRNA are the same nucleic acid construct. In some aspects, the nucleic acid construct that encodes the first gRNA and the nucleic acid construct that encodes the second gRNA are different nucleic acid constructs.

In some aspects, the nucleic acid construct that encodes the first gRNA and/or the nucleic acid construct that encodes the second gRNA is part of a viral vector. Thus, in some aspects, the administering comprises administering a viral vector comprising one or more of the nucleic acid construct that encodes the first gRNA and the nucleic acid construct that encodes the second gRNA.

In some aspects, the first gRNA comprises a first crRNA and the second gRNA comprises a second crRNA. In some aspects, at least a portion of the first crRNA is complementary to the first target sequence in the viral genome and wherein at least a portion of the second crRNA is complementary to the second target sequence in the viral genome.

In some aspects, the first gRNA comprises both a first crRNA and a first transactivating CRISPR RNA (tracrRNA). When the first crRNAand the first tracrRNA are present as a single transcript, the gRNA is referred to as a single guide RNA (sgRNA). Thus, the first gRNA can be referred to as a first sgRNA. In some aspects, the second gRNA comprises both a second crRNA and a second tracrRNA. Thus, the second gRNA can be referred to as a second sgRNA.

In some aspects, at least a portion of the first crRNA is complementary to at least a portion of the first tracrRNA and wherein at least a portion of the second crRNA is complementary to at least a portion of the second tracrRNA. In some aspects, the complementary sequences of the first crRNA and first tracrRNA hybridize. In some aspects, the complementary sequences of the second crRNA and second tracrRNA hybridize.

In some aspects, the disclosed methods further comprise a third gRNA, or a nucleic acid construct that encodes the third gRNA. In some aspects, the third gRNA comprises a tracrRNA. In some aspects, at least a portion of the tracrRNA is complementary to at least a portion of the first crRNA. Thus, in some aspects, the tracrRNA is a first tracrRNA. In some aspects, the first crRNA and the (first) tracrRNA being present on a sgRNA, the tracrRNA and crRNA can be on separate transcripts.

In some aspects, the disclosed methods comprise a fourth gRNA, or a nucleic acid construct that encodes the fourth gRNA. In some aspects, the fourth gRNA comprises a tracrRNA. In some aspects, at least a portion of the tracrRNA is complementary to at least a portion of the second crRNA. Thus, in some aspects, the tracrRNA is a second tracrRNA because it hybridizes to a second crRNA. Instead of the second crRNA and the (second) tracrRNA being present on a sgRNA, the tracrRNA and crRNA can be on separate transcripts.

In the methods disclosed herein, gRNAs can be designed to promote excision of the intervening regions of viral or proviral genomes. The cleavage pattern of Cas12d and Cas12e generates 5′ overhangs rather than blunt DNA double strand breaks characteristic of Cas9, which promotes the process of non-homologous end resection. In some aspects, the 5′ overhangs, and the presence of short sequences of homologous DNA sequences adjacent to the two or more Cas12d or Cas12e cut sites, can promote the excision of the intervening DNA through the cell's DNA repair processes. In some aspects, such processes can include direct joining of compatible overhangs or alternative end-joining processes. Promotion of end resection by 5′ overhangs can promote alternative end joining through processes termed microhomology-mediated end joining (MMEJ), which can also be known as alternative end joining, or alternative non-homologous end joining, or theta-mediated end joining (TMEJ). These processes can effectively excise the intervening DNA sequence that lies between two cut sites.

In some aspects, the methods disclosed herein can employ the identification of target regions in the genome or proviral genome of viruses for which a gRNA, and in some cases a second gRNA, and in some cases a third gRNA, can be designed in a manner to promote MMEJ or other cellular DNA repair mechanisms that utilize small regions of homology, known as microhomologies, for joining two or more cut sites to yield excision of regions of viral DNA disrupting viral replication or production of viral gene products. In some aspects, Cas12d or Cas12e gRNAs can be designed based on the viral DNA sequence(s): Either a specific sequence, or consensus sequence based on known viral sequences can be used. The viral sequence(s) can be scanned, either manually or using computational tools, to identify the location of PAM (protospacer adjacent motif) sequences. In some aspects, the PAMs for Cas12e enzymes can include TTCN where N is any nucleotide. In some aspects, the PAM for Cas12d enzymes can include TR, where R is a purine. In some aspects, the sequence of 16-23 base pairs 3′ to the PAM represents 5′ to the cut site at the 5′ end of the sequence to be excised, and 3′ to the cut site at the 3′ end of the sequence to be excised of the protospacer or potential target sequence.

In some aspects, the selection of gRNAs considers both the requirement for a PAM and the presence of microhomologies within, adjacent to, or in proximity to, the target site. Microhomologies consist of regions of homology, which may be from 3 or more nucleotides in length that occur more than once in a given viral sequence. In some aspects, when two or more gRNAs are used, the design considers the presence of the same region(s) of homology existing at individual gRNA targets. Further, consideration can be given to the position of the regions of homology relative to the DNA to be excised. That is, identified regions of homology can be located within or adjacent to the Cas12d or Cas12e gRNA target sites. To promote alternative end-joining microhomologies within the target site, the protospacer can be 5′ to the cut site, at the 5′ end of the sequence to be excised, and 3′ to the cut site at the 3′ end of the sequence to be excised. When located adjacent to the target site, the microhomologies can be 5′ to the target site, at the 5′ end of the sequence to be excised, and 3′ to the target site at the 3′ end of the sequence to be excised. In some aspects, topromote end joining through compatible 5′ overhangs, microhomology should exist within the region of the template strand and non-template strand cuts at each target site, producing complementary 5′ overhangs at each cut site.

### 2. Cas12d and Cas12e Endonucleases

Cas12d can also be referred to as CasY. Cas12e can also be referred to as CasX. In some aspects, the Cas X family includes, but is not limited to, CasX1 and CasX2. In some aspects, a CasY endonuclease can be, but is not limited to, CasY1-CasY1S. These Cas endonucleases generate staggered ends, which can also be referred to as 5′ overhangs, following cleavage.

A Cas12d or Cas12e polypeptide (this term is used interchangeably with the term “Cas12d or Cas12e endonuclease”) can bind and/or modify (e.g., cleave, nick, methylate, demethylate, etc.) a target nucleic acid and/or a polypeptide associated with target nucleic acid (e.g., methylation or acetylation of a histone tail) (e.g., in some cases the CasX protein includes a fusion partner with an activity, and in some cases the CasX protein provides nuclease activity). In some cases, the Cas12d or Cas12e protein is a naturally-occurring protein (e.g., naturally occurs in prokaryotic cells). In other cases, the Cas12d or Cas12e protein is not a naturally-occurring polypeptide (e.g., the Cas12d or Cas12e protein is a variant Cas12d or Cas12e protein, a chimeric protein, and the like).

A naturally occurring Cas12d or Cas12e protein functions as an endonuclease that catalyzes a double strand break at a specific sequence (e.g. target sequence) in a targeted double stranded DNA (dsDNA). The sequence specificity is provided by the associated guide RNA, which hybridizes to a target sequence within the target DNA. The naturally occurring guide RNA includes a tracrRNA hybridized to a crRNA, where the crRNA includes a guide sequence that hybridizes to a target sequence in the target DNA.

In some aspects, a variant Cas12d or Cas12e protein has an amino acid sequence that is different by at least one amino acid (e.g., has a deletion, insertion, substitution, fusion) when compared to the amino acid sequence of the corresponding wild type Cas12d or Cas12e protein.

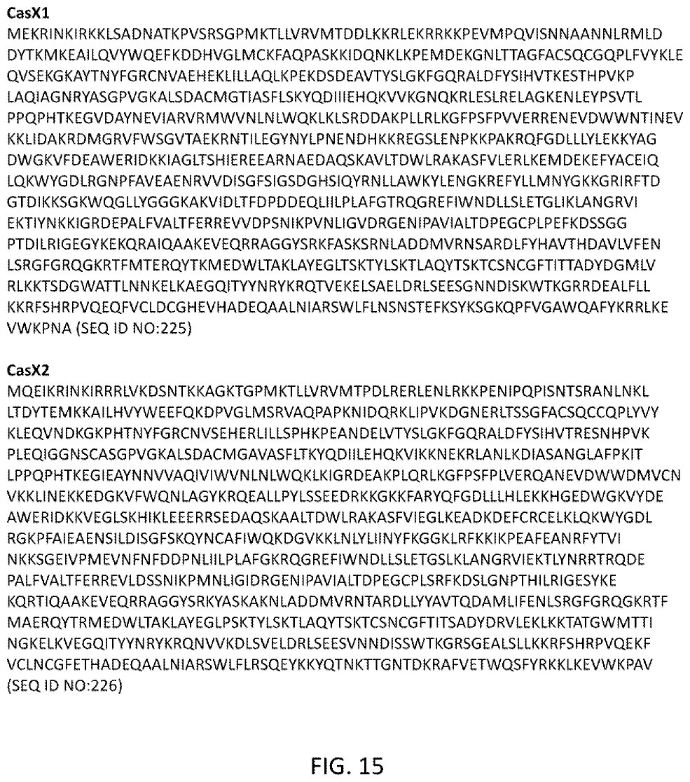

In some cases, a disclosed CasX protein includes an amino acid sequence having 20% or more sequence identity (e.g., 30% or more, 40% or more, 50% or more, 60% or more, 70% or more, 80% or more, 85% or more, 90% or more, 95% or more, 97% or more, 98% or more, 99% or more, or 100% sequence identity) with a wild type CasX protein sequence. Examples of Cas X protein sequences are shown in FIG. 15.

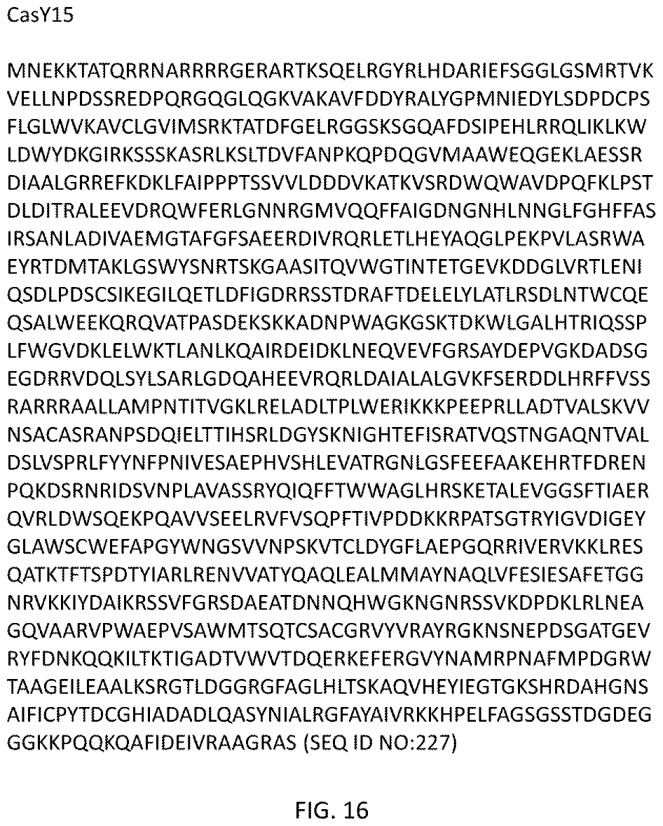

In some cases, a disclosed CasY protein includes an amino acid sequence having 20% or more sequence identity (e.g., 30% or more, 40% or more, 50% or more, 60% or more, 70% or more, 80% or more, 85% or more, 90% or more, 95% or more, 97% or more, 98% or more, 99% or more, or 100% sequence identity) with a wild type CasY protein sequence. Examples of Cas Y protein sequences are shown in FIG. 16.

In some aspects, a variant Cas12d or Cas12e endonuclease is a modified Cas12d or modified Cas12e, respectively. In some aspects, a modified Cas12d or Cas12e comprises a detectable label. In some aspects, the detectable label can be a chemiluminescent label, fluorescent label, or enzymatic label. As used herein, a “detectable label” is a nucleic acid, protein, or compound that can be detected or can lead to a detectable response. Detectable labels in accordance with the invention can be linked to a nucleic acid sequence or protein, such as Cas12d or Cas12e, either directly or indirectly, and include radioisotopes, enzymes, haptens, chromophores such as dyes or particles that impart a detectable color (e.g., latex beads or metal particles), luminescent compounds (e.g., bioluminescent, phosphorescent or chemiluminescent moieties), a quantum dot, and fluorescent compounds.

Suitable fluorescent proteins include, but are not limited to, green fluorescent protein (GFP) or variants thereof, blue fluorescent variant of GFP (BFP), cyan fluorescent variant of GFP (CFP), yellow fluorescent variant of GFP (YFP), enhanced GFP (EGFP), enhanced CFP (ECFP), enhanced YFP (EYFP), GFPS65T, Emerald, Topaz (TYFP), Venus, Citrine, mCitrine, GFPuv, destabilised EGFP (dEGFP), destabilised ECFP (dECFP), destabilised EYFP (dEYFP), mCFPm, Cerulean, T-Sapphire, CyPet, YPet, mKO, HcRed, t-HcRed, DsRed, DsRed2, DsRed-monomer, J-Red, dimer2, t-dimer2(12), mRFP1, pocilloporin, Renilla GFP, Monster GFP, paGFP, Kaede protein and kindling protein, Phycobiliproteins and Phycobiliprotein conjugates including B-Phycoerythrin, R-Phycoerythrin and Allophycocyanin. Other examples of fluorescent proteins include mHoneydew, mBanana, mOrange, dTomato, tdTomato, mTangerine, mStrawberry, mCherry, mGrapel, mRaspberry, mGrape2, mPlum (Shaner et al. (2005) Nat. Methods 2:905-909), and the like. Any of a variety of fluorescent and colored proteins from Anthozoan species, as described in, e.g., Matz et al. (1999) Nature Biotechnol. 17:969-973, is suitable for use.

Suitable enzymes include, but are not limited to, horse radish peroxidase (HRP), alkaline phosphatase (AP), beta-galactosidase (GAL), glucose-6-phosphate dehydrogenase, beta-N-acetylglucosaminidase, β-glucuronidase, invertase, Xanthine Oxidase, firefly luciferase, glucose oxidase (GO), and the like.

In some aspects, a modified Cas12d or Cas12e comprises a nuclear localization sequence. In some aspects, the presence of the nuclear localization sequence makes the Cas12d or Cas12e a modified Cas12d or Cas12e. In some aspects, the nucleic acid sequence that encodes the Cas12d or Cas12e comprises a nuclear localization sequence. In some aspects, a nuclear localization sequence can be a nucleic acid sequence or an amino acid sequence.

In some aspects, the sequence of Cas12d or Cas12e can be mutated. In some aspects, the mutation is not in a functional domain of Cas12d or Cas12e. In some aspects, domains that have required functions include the DNA binding domains consisting of a target-strand loading domain (e.g. Residues 825-934 of DpbCasX) and a non-target-strand binding domain (e.g. residues 101-191). Liu et al Nature, 566, 218-223 (2019), incorporated by reference herein, describes functional domains of CasX enzymes. Mutations that specifically abrogate CasX functions include those [e.g. D672A/E769A/D935A] targeting the RuvC domain.

In some aspects, the nucleic acid construct that encodes the Cas12d or Cas12e endonuclease is the same nucleic acid construct that encodes at least one of the first gRNA or second gRNA.

In some aspects, the nucleic acid construct that encodes the Cas12d or Cas12e endonuclease is part of a viral vector. Thus, in some aspects, the administering comprises administering a viral vector comprising one or more of the nucleic acid constructs that encodes the Cas12d or Cas12e endonuclease, the nucleic acid construct that encodes the first gRNA, and the nucleic acid construct that encodes the second gRNA.

In some aspects, the cell's DNA (e.g. DNA endogenous to the cell) is not cleaved by the Cas12d or Cas12e. Thus, for example, in some aspects, only the viral genome is cleaved by Cas12d or Cas12e in the disclosed methods, leaving the cell's DNA intact.

### 3. Virus

In some aspects, a cell comprises a cellular genome and the viral genome is integrated in the cellular genome. Thus, in some aspects a cell can comprise a single genome comprising both the cellular genome and the viral genome. In some aspects, the viral genome exists as an independent genomic element. Thus, in some aspects, a cell can comprise two separate genomes, the cellular genome and the viral genome.

In some aspects, the cell can be a mammalian cell. For example, in some aspects, the mammalian cell can be a human cell. Thus, in some aspects, the virus has infected a human cell.

In some aspects, the virus has a double stranded DNA (dsDNA) genome or a viral replication intermediate comprised of dsDNA. For example, RNA viruses (viruses with an RNA viral genome) can convert the RNA to DNA which can then be copied into dsDNA (e.g. a viral replication intermediate comprised of dsDNA). RNA viruses can include, but are not limited to, those of the family Retrovirales, including the subfamily Lentivirus (e.g. HIV-1 and HIV-2), Deltaretroviridae (e.g. HTLV-1 and HTLV-2), as well as the family Spumaretrovirinae (e.g. HFV) and Gammaretrovirinae (e.g. XMRV).

In some aspects, the disclosed methods inactivate a virus wherein the virus is from the family Retroviridae, Hepadnaviridae, Herpesviridae, Adenoviridae, Papillomaviridae, Poxviridae, Polyomaviridae, Asfarviridae. In some aspects, family Hepadnaviridae, can include Hepatitis B Virus (HBV). In some aspects, the virus can be from the order Herpesvirales, including pathogenic members of the families Alphaherpesvirinae, Betaherpesvirinae, and Gammaherpesvirinae. In some aspects, examples of viruses are as follows: members of the family Adenoviridae (e.g. Human Adenovirus species A through G), Papillomaviridae (e.g. Human Papilloma Viruses), Poxviridae (e.g. parpapoxvirus, orthopoxvirus, molluscipoxvirus), Polyomaviridae (e.g. Human Polyomavirus 1-14), and Asfarviridae (e.g. African Swine Fever Virus). In particular, in some aspects, the virus is HIV-1, Hepatitis B virus (HBV), or HTLV-1.

In some aspects, at least a portion of the viral genome is excised from the rest of the viral genome. In some aspects, all of the viral genes are excised from the viral genome. For example, when a gRNA cleaves at target sequences in an LTR region the all of the viral genes can be excised.

### 4. Target Sequences

In some aspects, the target sequence is the site on the target nucleic acid (e.g. viral genome) being targeted by the gRNA and the Cas12d or Cas12e for cleavage.

In some aspects, at least one of the first target sequence or second target sequence is a sequence from Table 1.

TABLE 1
Examples of Cas12e Target Sequences in HBV. Position and sequence as in HBV
strain ayw (NC_003977). Sequence begins with the first base of the PAM sequence,
and includes the PAM (TTCN) followed by the 20 bp protospacer sequence.

| Sense Strand | | Antisense Strand | |
|---|---|---|---|
| Position | | Position | |
| of first | | of first | |
| base of | | base of | |
| PAM | Sequence | PAM | Sequence |
| 3 | Ttccacaaccttccaccaaactct | 82 | Ttcctgaactggagccaccagcag |
| | (SEQ ID NO: 1) | | (SEQ ID NO: 2) |
| 13 | Ttccaccaaactctgcaagatccc | 155 | Ttcagcgcagggtccccaatcctc |
| | (SEQ ID NO: 3) | | (SEQ ID NO: 4) |
| 55 | Ttccctgctggtggctccagttca | 164 | Ttctccatgttcagcgcagggtcc |
| | (SEQ ID NO: 5) | | (SEQ ID NO: 6) |
| 75 | Ttcaggaacagtaaaccctgttct | 229 | Ttcttgtcaacaagaaaaaccccg |
| | (SEQ ID NO: 7) | | (SEQ ID NO: 8) |
| 95 | Ttctgactactgcctctcccttat | 289 | Ttccccctagaaaattgagagaag |
| | (SEQ ID NO: 9) | | (SEQ ID NO: 10) |
| 127 | Ttctcgaggattggggaccctgcg | 547 | Ttccttgagcagtagtcatgcagg |
| | (SEQ ID NO: 11) | | (SEQ ID NO: 12) |
| 178 | Ttcctaggaccccttctcgtgtta | 592 | Ttccgtccgaaggtttggtacagc |
| | (SEQ ID NO: 13) | | (SEQ ID NO: 14) |
| 191 | Ttctcgtgttacaggcggggtttt | 634 | Ttccgaaagcccaggatgatggga |
| | (SEQ ID NO: 15) | | (SEQ ID NO: 16) |
| 214 | Ttcttgttgacaagaatcctcaca | 920 | Ttcttgtggcaaggacccataaca |
| | (SEQ ID NO: 17) | | (SEQ ID NO: 18) |
| 267 | Ttctctcaattttctagggggaac | 944 | Ttctttgattttttgtatgatgtg |
| | (SEQ ID NO: 19) | | (SEQ ID NO: 20) |
| 278 | Ttctagggggaactaccgtgtgtc | 954 | Ttctaaaacattctttgatttttt |
| | (SEQ ID NO: 21) | | (SEQ ID NO: 22) |
| 312 | Ttcgcagtccccaacctccaatca | 985 | Ttccaatcaataggcctgttaata |
| | (SEQ ID NO: 23) | | (SEQ ID NO: 24) |
| 403 | Ttcctcttcatcctgctgctatgc | 999 | Ttcgttgacatactttccaatcaa |
| | (SEQ ID NO: 25) | | (SEQ ID NO: 26) |
| 409 | Ttcatcctgctgctatgcctcatc | 1141 | Ttcaggtattgtttacacagaaag |
| | (SEQ ID NO: 27) | | (SEQ ID NO: 28) |
| 433 | Ttcttgttggttcttctggactat | 1245 | Ttccacgcatgcgctgatggccca |
| | (SEQ ID NO: 29) | | (SEQ ID NO: 30) |
| 443 | Ttcttctggactatcaaggtatgt | 1280 | Ttccgcagtatggatcggcagagg |
| | (SEQ ID NO: 31) | | (SEQ ID NO: 32) |
| 446 | Ttctggactatcaaggtatgttgc | 1447 | Ttcagcgccgacgggacgtaaaca |
| | (SEQ ID NO: 33) | | (SEQ ID NO: 34) |
| 485 | Ttccaggatcctcaacaaccagca | 1626 | ttcacggtggtctccatgcgacgt |
| | (SEQ ID NO: 35) | | (SEQ ID NO: 36) |
| 582 | Ttcggacggaaattgcacctgtat | 1926 | Ttctttataagggtcgatgtccat |
| | (SEQ ID NO: 37) | | (SEQ ID NO: 38) |
| 605 | Ttcccatcccatcatcctgggctt | 2022 | Ttcccgatacagagctgaggcggt |
| | (SEQ ID NO: 39) | | (SEQ ID NO: 40) |
| 628 | Ttcggaaaattcctatgggagtgg | 2094 | Ttccccccagcaaagaattgcttg |
| | (SEQ ID NO: 41) | | (SEQ ID NO: 42) |
| 637 | Ttcctatgggagtgggcctcagcc | 2133 | Ttccaaattaacacccacccaggt |
| | (SEQ ID NO: 43) | | (SEQ ID NO: 44) |
| 664 | Ttctcctggctcagtttactagtg | 2236 | Ttccaaaagtgagacaagaaatgt |
| | (SEQ ID NO: 45) | | (SEQ ID NO: 46) |
| 695 | Ttcagtggttcgtagggctttccc | 2241 | Ttctcttccaaaagtgagacaaga |
| | (SEQ ID NO: 47) | | (SEQ ID NO: 48) |
| 703 | Ttcgtagggctttcccccactgtt | 2374 | Ttctaggggacctgcctcgtcgtc |
| | (SEQ ID NO: 49) | | (SEQ ID NO: 50) |
| 714 | Ttcccccactgtttggctttcagt | 2377 | Ttcttctaggggacctgcctcgtc |
| | (SEQ ID NO: 51) | | (SEQ ID NO: 52) |
| 732 | Ttcagttatatggatgatgtggta | 2380 | Ttcttcttctaggggacctgcctc |
| | (SEQ ID NO: 53) | | (SEQ ID NO: 54) |
| 811 | Ttcttttgtctttgggtatacatt | 2401 | Ttcgtctgcgaggcgagggagttc |
| | (SEQ ID NO: 55) | | (SEQ ID NO: 56) |
| 958 | Ttcctattaacaggcctattgatt | 2425 | Ttctgcgacgcggcgattgagacc |
| | (SEQ ID NO: 57) | | (SEQ ID NO: 58) |
| 1075 | Ttcaatctaagcaggctttcactt | 2442 | Ttcccgagattgagatcttctgcg |
| | (SEQ ID NO: 59) | | (SEQ ID NO: 60) |
| 1092 | Ttcactttctcgccaacttacaag | 2481 | Ttccccaccttatgagtccaagga |
| | (SEQ ID NO: 61) | | (SEQ ID NO: 62) |
| 1098 | Ttctcgccaacttacaaggccttt | 2532 | Ttccaatgaggattaaagacaggt |
| | (SEQ ID NO: 63) | | (SEQ ID NO: 64) |
| 1120 | Ttctgtgtaaacaatacctgaacc | 2587 | Ttcacattttttgataatgtcttg |
| | (SEQ ID NO: 65) | | (SEQ ID NO: 66) |
| 1250 | Ttcggctcctctgccgatccatac | 2619 | Ttctcattaactgtgagtgggcct |
| | (SEQ ID NO: 67) | | (SEQ ID NO: 68) |
| 1372 | Ttccatggctgctaggctgtgctg | 2624 | Ttcttttctcattaactgtgagtg |
| | (SEQ ID NO: 69) | | (SEQ ID NO: 70) |
| 1463 | Ttctcggggtcgcttgggactctc | 2713 | Ttctggataataaggtttaatacc |
| | (SEQ ID NO: 71) | | (SEQ ID NO: 72) |
| 1495 | Ttctccgtctgccgttccgaccga | 2766 | Ttccatagagtgtgtaaatagtgt |
| | (SEQ ID NO: 73) | | (SEQ ID NO: 74) |
| 1509 | Ttccgaccgaccacggggcgcacc | 2791 | Ttctctcttatataatatacccgc |
| | (SEQ ID NO: 75) | | (SEQ ID NO: 76) |
| 1562 | Ttctcatctgccggaccgtgtgca | 2836 | Ttcccaagaatatggtgacccaca |
| | (SEQ ID NO: 77) | | (SEQ ID NO: 78) |
| 1587 | Ttcgcttcacctctgcacgtcgca | 2860 | Ttctgccccatgctgtagatcttg |
| | (SEQ ID NO: 79) | | (SEQ ID NO: 80) |
| 1592 | Ttcacctctgcacgtcgcatggag | 3123 | Ttcctgactggcgattggtggagg |
| | (SEQ ID NO: 81) | | (SEQ ID NO: 82) |
| 1709 | Ttcaaagactgtttgtttaaagac | 3156 | Ttctcaaaggtggagacagcgggg |
| | (SEQ ID NO: 83) | | (SEQ ID NO: 84) |
| 1826 | Ttcacctctgcctaatcatctctt | | |
| | (SEQ ID NO: 85) | | |
| 1851 | Ttcatgtcctactgttcaagcctc | | |
| | (SEQ ID NO: 86) | | |
| 1865 | Ttcaagcctccaagctgtgccttg | | |
| | (SEQ ID NO: 87) | | |
| 1962 | Ttctgacttctttccttcagtacg | | |
| | (SEQ ID NO: 88) | | |
| 1969 | Ttctttccttcagtacgagatctt | | |
| | (SEQ ID NO: 89) | | |
| 1973 | Ttccttcagtacgagatcttctag | | |
| | (SEQ ID NO: 90) | | |
| 1977 | Ttcagtacgagatcttctagatac | | |
| | (SEQ ID NO: 91) | | |
| 1991 | Ttctagataccgcctcagctctgt | | |
| | (SEQ ID NO: 92) | | |
| 2046 | Ttcacctcaccatactgcactcag | | |
| | (SEQ ID NO: 93) | | |
| 2078 | Ttctttgctggggggaactaatga | | |
| | (SEQ ID NO: 94) | | |
| 2191 | Ttcaggcaactcttgtggtttcac | | |
| | (SEQ ID NO: 95) | | |
| 2210 | Ttcacatttcttgtctcacttttg | | |
| | (SEQ ID NO: 96) | | |
| 2217 | Ttcttgtctcacttttggaagaga | | |
| | (SEQ ID NO: 97) | | |
| 2266 | Ttcggagtgtggattcgcactcct | | |
| | (SEQ ID NO: 98) | | |
| 2279 | Ttcgcactcctccagcttatagac | | |
| | (SEQ ID NO: 99) | | |
| 2330 | Ttccggagactactgttgttagac | | |
| | (SEQ ID NO: 100) | | |
| 2457 | Ttccttggactcataaggtgggga | | |
| | (SEQ ID NO: 101) | | |
| 2497 | Ttcttctactgtacctgtctttaa | | |
| | (SEQ ID NO: 102) | | |
| 2500 | Ttctactgtacctgtctttaatcc | | |
| | (SEQ ID NO: 103) | | |
| 2544 | Ttcctaatatacatttacaccaag | | |
| | (SEQ ID NO: 104) | | |
| 2732 | Ttccaaactagacactatttacac | | |
| | (SEQ ID NO: 105) | | |
| 2827 | Ttcttgggaacaagatctacagca | | |
| | (SEQ ID NO: 106) | | |
| 2864 | Ttccaccagcaatcctctgggatt | | |
| | (SEQ ID NO: 107) | | |
| 2886 | Ttctttcccgaccaccagttggat | | |
| | (SEQ ID NO: 108) | | |
| 2890 | Ttcccgaccaccagttggatccag | | |
| | (SEQ ID NO: 109) | | |
| 2916 | Ttcagagcaaacaccgcaaatcca | | |
| | (SEQ ID NO: 110) | | |
| 2949 | Ttcaatcccaacaaggacacctgg | | |
| | (SEQ ID NO: 111) | | |
| 3003 | Ttcgggctgggtttcaccccaccg | | |
| | (SEQ ID NO: 112) | | |
| 3015 | Ttcaccccaccgcacggaggcctt | | |

(SEQ ID NO: 113)

In some aspects, the first target sequence and second target sequence are one of the pairs of target sequences provided in Table 2.

TABLE 2
Examples of Paired gRNA for Excision of
portions of viral genomes. Sequences denote
the protospacer sequences within different viruses.

| | | | |
|---|---|---|---|
| Strand | Protospacer | Strand | Protospacer |

HBV strain ayw (NC_003977)

| | | | |
|---|---|---|---|
| antisense | Tctaggggacctgcctcgtc | sense | Gagctactgtggagttactc |
| | (SEQ ID NO: 114) | | (SEQ ID NO: 115) |
| antisense | Ccaccttatgagtccaagga | sense | Tcctgctgctatgcctcatc |
| | (SEQ ID NO: 116) | | (SEQ ID NO: 117) |
| antisense | Cattttttgataatgtcttg | sense | Gttatatggatgatgtggta |
| | (SEQ ID NO: 118) | | (SEQ ID NO: 119) |
| antisense | Cattaactgtgagtgggcct | sense | Gtggttcgtagggctttccc |
| | (SEQ ID NO: 120) | | (SEQ ID NO: 121) |
| antisense | Atagagtgtgtaaatagtgt | sense | Gttatatggatgatgtggta |
| | (SEQ ID NO: 122) | | (SEQ ID NO: 123) |
| antisense | Ctcttatataatatacccgc | sense | Tgttggttcttctggactat |
| | (SEQ ID NO: 124) | | (SEQ ID NO : 125) |
| antisense | Ctcttatataatatacccgc | sense | Gttatatggatgatgtggta |
| | (SEQ ID NO: 126) | | (SEQ ID NO: 127) |
| antisense | Gccccatgctgtagatcttg | sense | Gacttctttccttcagtacg |
| | (SEQ ID NO: 128) | | (SEQ ID NO: 129) |
| antisense | Actgcatggcctgaggatga | sense | Tcttcatcctgctgctatgc |
| | (SEQ ID NO: 130) | | (SEQ ID NO: 131) |
| antisense | Actgcatggcctgaggatga | sense | Gaaaattcctatgggagtgg |
| | (SEQ ID NO: 132) | | (SEQ ID NO: 133) |

HIV_HXB2

| | | | |
|---|---|---|---|
| antisense | Gctaatcagggaagtagcc | sense | Gcattatcagaaggagccac |
| | (SEQ ID NO: 134) | | (SEQ ID NO: 135) |

HBV_B

| | | | |
|---|---|---|---|
| antisense | Ataagattgacgatatggca | sense | Catctgccggaccgtgtgcac |
| | (SEQ ID NO: 136) | | (SEQ ID NO: 137) |
| HTLV1 | | | |
| antisense | Gggatagtgggctttaggcg | sense | Ttaataccgaacccagccaa |
| | (SEQ ID NO: 138) | | (SEQ ID NO: 139) |

JCV

| | | | |
|---|---|---|---|
| antisense | Tttctggtgggatcaggaac | sense | Gataagcttttctcatgaca |
| | (SEQ ID NO: 140) | | (SEQ ID NO: 141) |

In some aspects, at least one of the first target sequence or second target sequence is a sequence from Table 2.

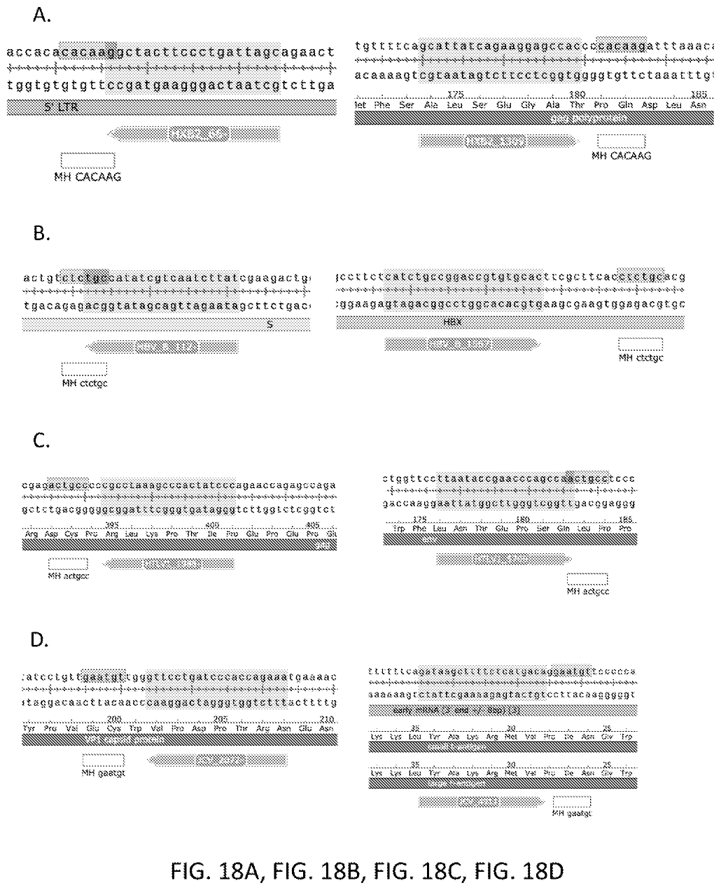

Table 2 provides examples of Cas12e target sequences in a HBV Clade A consensus. Position and sequence as in HBV clade A consensus sequence are provided herein (FIG. 18). In some aspects, the sequence can begin with the first base of a PAM sequence, and includes the PAM (TTCN) followed by the 20 bp protospacer sequence.

In some aspects, the first target sequence and second target sequence are on opposite strands of the viral genome.

In some aspects, the cleavage at the first target sequence and the second target sequence results in 5′ single stranded DNA (ssDNA) overhangs at the first target sequence and the second target sequence. Thus, in some aspects, cleavage by Cas12d or Cas12e does not result in blunt ends.

In some aspects, the 5′ ssDNA overhangs at the first target sequence and the second target sequence have complementary overlapping sequences. In some aspects, the methods further comprise the 5′ ssDNA overhangs at the first target sequence and the second target sequence hybridize with each other.

In some aspects, sequences adjacent to the 5′ssDNA overhangs at the first target sequence and the second target sequence are of homologous sequence. In some aspects, the methods disclosed herein further comprise the joining of these regions of short homology through DNA repair mechanisms that utilize these regions of homology, for example including microhomology mediated end joining. In some aspects, the methods disclosed herein further comprise the removal of the intervening DNA sequence following the joining of these regions of short homology through DNA repair mechanisms present in eukaryotic cells that utilize these regions of homology including microhomology mediated end joining. In some aspects, microhomology-mediated end joining (MMEJ) is an error-prone repair mechanism that involves alignment of microhomologous sequences internal to the broken ends before joining, and is associated with deletions and insertions that mark the original break site, as well as chromosome translocations.

### C. Compositions

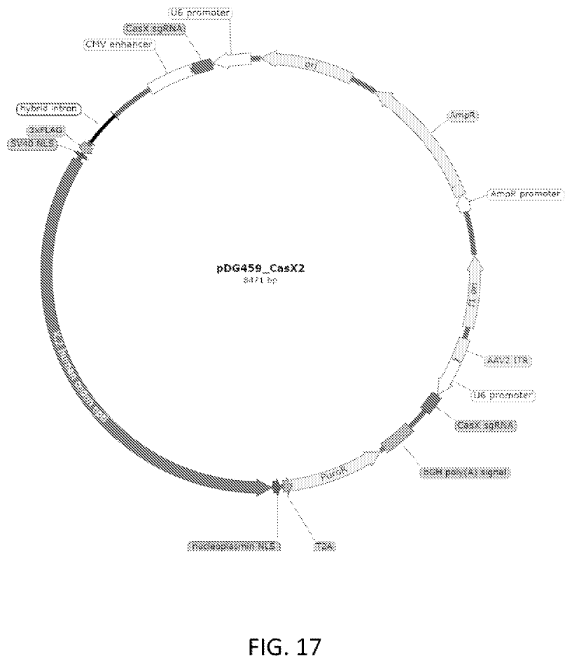

Disclosed are constructs used to deliver CasX or CasY (e.g. Cas12d or Cas12e) into cells. For example, FIG. 17 shows a vector carrying CasX2. Disclosed are constructs used to deliver any of the gRNAs disclosed herein into cells. For example, any of the gRNAs described herein can be present in a construct, alone or in combination with a second gRNA or with a nucleic acid encoding a CasX or CasY endonuclease.

Disclosed are gRNAs used to target sequences in the HBV genome. Disclosed herein are gRNAs that target the HBV genome including, but not limited to: Hbv 353—tctaggggacctgcctcgtc (SEQ ID NO: 142); Hbv 457—ccaccttatgagtccaagga (SEQ ID NO:143); Hbv 688—ggtattaaaccttattatcc (SEQ ID NO: 144); Hbv 835—caagatctacagcatggggc (SEQ ID NO:144); Hbv 1446—ccctagaaaattgagagaag (SEQ ID NO:145); Hbv1704—ttgagcagtagtcatgcagg (SEQ ID NO: 146); Hbv 1791—gaaagcccaggatgatggga (SEQ ID NO:147); Hbv 2101—ttgattttttgtatgatgtg (SEQ ID NO: 148); Hbv 2665—cggggtcgcttgggactctc (SEQ ID NO:149); Hbv 2789—cttcacctctgcacgtcgca (SEQ ID NO: 150); Hbv 2794 (Hbx216)—cctctgcacgtcgcatggag (SEQ ID NO:151); Hbv 2911 (Hbx333)—aagactgtttgtttaaagac (SEQ ID NO:152); Hbv 3164—gacttctttccttcagtacg (SEQ ID NO:153); Hbx 91 (Hbv 2665)—cggggtcgcttgggactctc (SEQ ID NO:154); Hbx 119 (Hbv 2697)—ccgtctgccgttccgaccga (SEQ ID NO:155); Hbx 137 (Hbv 2711)—gaccgaccacggggcgcacc (SEQ ID NO:156); Hbx 186 (Hbv 2764)—catctgccggaccgtgtgca (SEQ ID NO:157); Hbv 99—ctgatgacggagagggaata (SEQ ID NO:158); Hbv 131—gctcctaacccctgggacgc (SEQ ID NO:159); hbv 182—atcctggggaagagcacaat (SEQ ID NO: 160); hbv 195—gcacaatgtccgccccaaaa (SEQ ID NO:161); hbv 1499—ccgtctgccgttccgaccga (SEQ ID NO:162); hbv 1513—gaccgaccacggggcgcacc (SEQ ID NO:163); hbv 1566—catctgccggaccgtgtgca (SEQ ID NO:164); hbv 1596—cctctgcacgtcgcatggag (SEQ ID NO:165); hbv 1713—aagactgtttgtttaaagac (SEQ ID NO:166); hbv 1903—aatattcccagctacaggta (SEQ ID NO: 167); hbv 1966—ctgaagaaaggaagtcatgc (SEQ ID NO:168); hbv 1973—aaggaagtcatgctctagaa (SEQ ID NO:169); hbv 1977—aagtcatgctctagaagatc (SEQ ID NO: 170); hbv 1981—catgctctagaagatctatg (SEQ ID NO:171); hbv 1995—tctatggcggagtcgagaca (SEQ ID NO:172).

A CasX protein binds to target DNA at a target sequence defined by the region of complementarity between the DNA-targeting RNA (gRNA) and the target DNA. As is the case for many CRISPR endonucleases, site-specific binding (and/or cleavage) of a double stranded target DNA occurs at locations determined by both (i) base-pairing complementarity between the guide RNA and the target DNA; and (ii) a short motif [referred to as the protospacer adjacent motif (PAM)] in the target DNA.

In some embodiments, the PAM for a CasX protein is immediately 5′ of the target sequence of the non-complementary strand of the target DNA (the complementary strand hybridizes to the guide sequence of the guide RNA while the non-complementary strand does not directly hybridize with the guide RNA and is the reverse complement of the non-complementary strand). In some cases, different CasX proteins (i.e., CasX proteins from various species) may be advantageous to use in the various provided methods in order to capitalize on various enzymatic characteristics of the different CasX proteins (e.g., for different PAM sequence preferences; for increased or decreased enzymatic activity; for an increased or decreased level of cellular toxicity; to change the balance between NHEJ, homology-directed repair, single strand breaks, double strand breaks, etc.; to take advantage of a short total sequence; and the like). CasX proteins from different species may require different PAM sequences in the target DNA. Various methods (including in silico and/or wet lab methods) for identification of the appropriate PAM sequence are known in the art and are routine, and any convenient method can be used.

### 1. Pharmaceutical Compositions

Disclosed are compositions comprising a Cas12d or Cas12e endonuclease, a nucleic acid construct that encodes the Cas12d or Cas12e endonuclease, a first gRNA, a nucleic acid construct that encodes the first gRNA, a second gRNA, and/or a nucleic acid construct that encodes the second gRNA.

In some aspects, the disclosed compositions can be pharmaceutical compositions. For example, in some aspects, disclosed are pharmaceutical compositions comprising a composition comprising one or more of the gRNAs disclosed herein and a pharmaceutically acceptable carrier. By “pharmaceutically acceptable” is meant a material or carrier that would be selected to minimize any degradation of the active ingredient and to minimize any adverse side effects in the subject, as would be well known to one of skill in the art. Examples of carriers include dimyristoylphosphatidyl (DMPC), phosphate buffered saline or a multivesicular liposome. For example, PG:PC:Cholesterol:peptide or PC:peptide can be used as carriers in this invention. Other suitable pharmaceutically acceptable carriers and their formulations are described in Remington: The Science and Practice of Pharmacy (19th ed.) ed. A.R. Gennaro, Mack Publishing Company, Easton, PA 1995. Typically, an appropriate amount of pharmaceutically-acceptable salt is used in the formulation to render the formulation isotonic. Other examples of the pharmaceutically-acceptable carrier include, but are not limited to, saline, Ringer's solution and dextrose solution. The pH of the solution can be from about 5 to about 8, or from about 7 to about 7.5. Further carriers include sustained release preparations such as semi-permeable matrices of solid hydrophobic polymers containing the composition, which matrices are in the form of shaped articles, e.g., films, stents (which are implanted in vessels during an angioplasty procedure), liposomes or microparticles. It will be apparent to those persons skilled in the art that certain carriers may be more preferable depending upon, for instance, the route of administration and concentration of composition being administered, and the organ or cell type that is targeted for therapy. These most typically would be standard carriers for administration of drugs to humans, including solutions such as sterile water, saline, and buffered solutions at physiological pH.

Pharmaceutical compositions can also include carriers, thickeners, diluents, buffers, preservatives and the like, as long as the intended activity of the polypeptide, peptide, or conjugate of the invention is not compromised. Pharmaceutical compositions may also include one or more active ingredients (in addition to the composition of the invention) such as antimicrobial agents, anti-inflammatory agents, anesthetics, and the like.

The pharmaceutical compositions as disclosed herein can be prepared for oral or parenteral administration. Pharmaceutical compositions prepared for parenteral administration include those prepared for intravenous (or intra-arterial), intramuscular, subcutaneous, intraperitoneal, transmucosal (e.g., intranasal, intravaginal, or rectal), or transdermal (e.g., topical) administration. Aerosol inhalation can also be used to deliver the fusion proteins. Thus, compositions can be prepared for parenteral administration that includes fusion proteins dissolved or suspended in an acceptable carrier, including but not limited to an aqueous carrier, such as water, buffered water, saline, buffered saline (e.g., PBS), and the like. One or more of the excipients included can help approximate physiological conditions, such as pH adjusting and buffering agents, tonicity adjusting agents, wetting agents, detergents, and the like. Where the compositions include a solid component (as they may for oral administration), one or more of the excipients can act as a binder or filler (e.g., for the formulation of a tablet, a capsule, and the like). Where the compositions are formulated for application to the skin or to a mucosal surface, one or more of the excipients can be a solvent or emulsifier for the formulation of a cream, an ointment, and the like.

Preparations of parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for optical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids, or binders may be desirable. Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mon-, di-, trialkyl and aryl amines and substituted ethanolamines.

The pharmaceutical compositions can be sterile and sterilized by conventional sterilization techniques or sterile filtered. Aqueous solutions can be packaged for use as is, or lyophilized, the lyophilized preparation, which is encompassed by the present disclosure, can be combined with a sterile aqueous carrier prior to administration. The pH of the pharmaceutical compositions typically will be between 3 and 11 (e.g., between about 5 and 9) or between 6 and 8 (e.g., between about 7 and 8). The resulting compositions in solid form can be packaged in multiple single dose units, each containing a fixed amount of the above-mentioned agent or agents, such as in a sealed package of tablets or capsules. The composition in solid form can also be packaged in a container for a flexible quantity, such as in a squeezable tube designed for a topically applicable cream or ointment.

The pharmaceutical compositions described above can be formulated to include a therapeutically effective amount of a composition disclosed herein. In some aspects, therapeutic administration encompasses prophylactic applications. Based on genetic testing and other prognostic methods, a physician in consultation with their patient can choose a prophylactic administration where the patient has a clinically determined predisposition or increased susceptibility (in some cases, a greatly increased susceptibility) to one or more autoimmune diseases or where the patient has a clinically determined predisposition or increased susceptibility (in some cases, a greatly increased susceptibility) to cancer.

The pharmaceutical compositions described herein can be administered to the subject (e.g., a human subject or human patient) in an amount sufficient to delay, reduce, or preferably prevent the onset of clinical disease. Accordingly, in some aspects, the subject is a human subject. In therapeutic applications, compositions are administered to a subject (e.g., a human subject) already with or diagnosed with a disease in an amount sufficient to at least partially improve a sign or symptom or to inhibit the progression of (and preferably arrest) the symptoms of the condition, its complications, and consequences. An amount adequate to accomplish this is defined as a “therapeutically effective amount.” A therapeutically effective amount of a pharmaceutical composition can be an amount that achieves a cure, but that outcome is only one among several that can be achieved. As noted, a therapeutically effective amount includes amounts that provide a treatment in which the onset or progression of the cancer is delayed, hindered, or prevented, or the autoimmune disease or a symptom of the autoimmune disease is ameliorated. One or more of the symptoms can be less severe. Recovery can be accelerated in an individual who has been treated.

The total effective amount of the conjugates in the pharmaceutical compositions disclosed herein can be administered to a mammal as a single dose, either as a bolus or by infusion over a relatively short period of time, or can be administered using a fractionated treatment protocol in which multiple doses are administered over a more prolonged period of time (e.g., a dose every 4-6, 8-12, 14-16, or 18-24 hours, or every 2-4 days, 1-2 weeks, or once a month). Alternatively, continuous intravenous infusions sufficient to maintain therapeutically effective concentrations in the blood are also within the scope of the present disclosure.

The pharmaceutical composition may be administered in a number of ways depending on whether local or systemic treatment is desired, and on the area to be treated.

### D. Kits

The materials described above as well as other materials can be packaged together in any suitable combination as a kit useful for performing, or aiding in the performance of, the disclosed method. It is useful if the kit components in a given kit are designed and adapted for use together in the disclosed method. For example, disclosed are kits comprising one or more of the disclosed gRNAs, Cas12d, or Cas12e, The kits also can contain vectors (e.g. nucleic acid constructs).

### Examples

### A. Example 1

An approach has been developed that leverages the mechanism of cutting of CasX, also known as Cas12e, family of RNA-guided endonucleases to mediate targeted disruption or deletion of portions of the Hepatitis B virus (HBV) genome. These methods apply to deletion of portions of the HBV genome that have integrated within the host or model system genomic DNA or that exist within cells as an independent genomic element such as a covalently closed circular DNA (cccDNA). Disclosed herein is a modified CasX enzyme used with an RNA based guide RNA (gRNA) consisting of either a transactivating CRISPR RNA (tracrRNA) and CRIPSR RNA (crRNA), or single guide RNA (sgRNA) comprised of tracrRNA and crRNA. Further described is the use of gRNA or gRNA combinations that, through consideration of sequences present at the sites of DNA cleavage induced by CasX and two or more gRNAs, promote excision of the intervening HBV DNA sequence. This example describes several CasX and gRNA combinations that achieve efficient, targeted excision of essential portions of the HBV genome. It should be recognized that Cas12d can also be used and other viral genomes, in addition to HBV, can be targeted.

CasX comprises a recently discovered group of RNA-guided endonucleases that mediate staggered cuts in dsDNA. In contrast to Cas9, which produces dsDNA breaks resulting in blunt-ended DNA, CasX produces staggered cuts resulting in 5′ ssDNA overhangs comprised of 5 or more bp of ssDNA.

Cas9-based strategies are being explored to mediate excision of the HBV genome. These approaches, whether using SaCas9 or SpCas9 are limited by pre-existing human immunity to these enzymes, and also the ability of the virus to evolve resistance to Cas9 gRNA approaches through mutation of sites within the protospacer sequence or protospacer adjacent motif (PAM). A CasX-based strategy has been developed that addresses these key limitations of existing approaches. CasX offers distinct advantages including relatively small size and presumed lack of the pre-existing immunity.

Through the design and use of appropriately paired CasX sgRNAs targeting HBV, this approach achieves highly efficient, targeted excision of large segments of the HBV proviral genome (Table 1).

Multiple CasX target sites (target sequences) have been identified within the HBV genome (shown in Table 1).

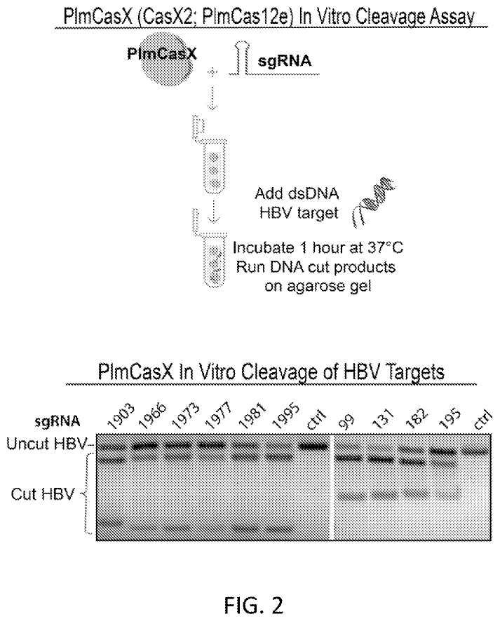

The tools to produce and utilize multiple sgRNAs that can function with CasX to cleave HBV dsDNA sequences in vitro have been identified (FIG. 2). These tools include vectors encoding various forms of CasX1 or CasX2 with human or E. coli optimized codon usage, and various tags to enhance expression, folding and purification of the protein after expression in E. coli. CasX1 and CasX2 are different endonucleases that can use the same gRNAs during the disclosed methods.

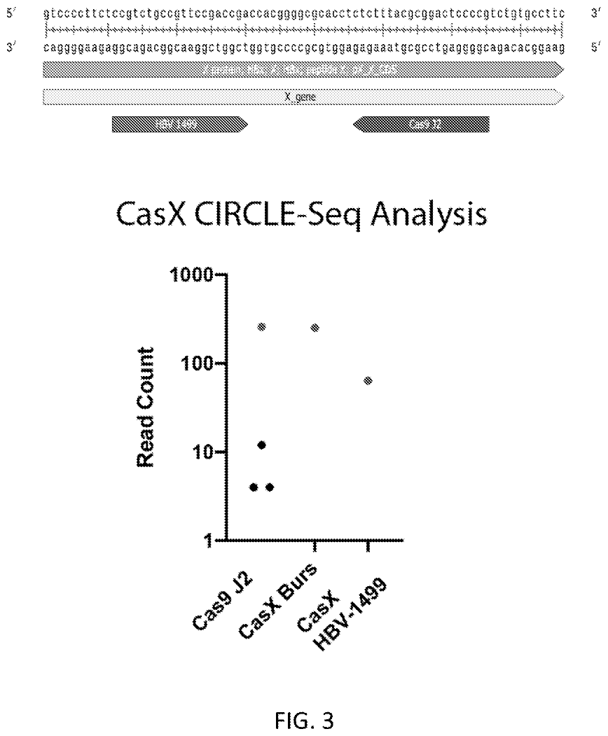

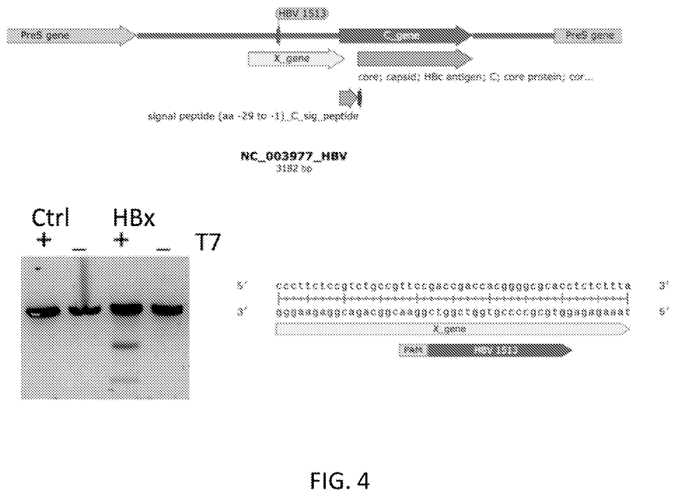

Highly specific cleavage of HBV dsDNA without detectable cleavage of host genomic DNA (FIG. 3) is achieved using modified forms of CasX1 or CasX2, containing combinations of the nuclear localization sequences Sv40 and nucleoplasmin and epitope tags to follow expression and subcellular localization of CasX proteins expressed in human or other mammalian cells.

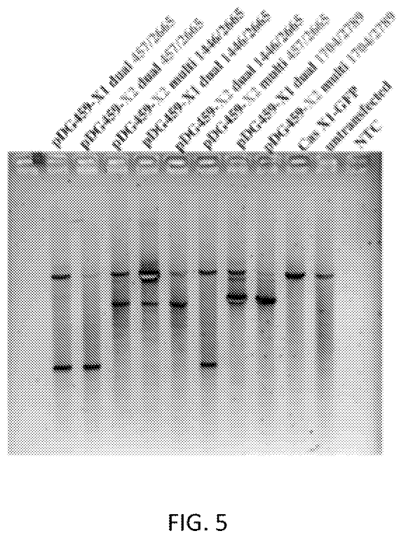

Tools have been developed for the delivery of CasX encoding constructs along with sgRNA targeting HBV, in human cells. Using these, HBV DNA integrating into the human genome was disrupted (See FIG. 5).

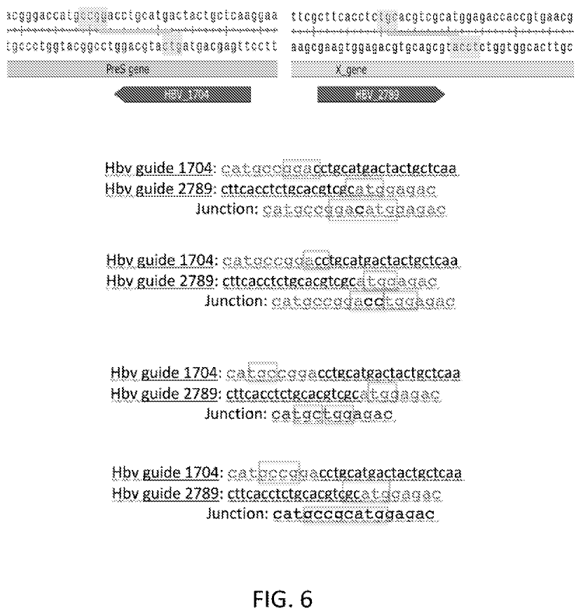

Paired sgRNA have been developed that target sequences on opposite strands of the HBV genome (Table 2) and result in efficient target cleavage with removal of intervening segments of HBV DNA in human cells (FIGS. 5 and 6).

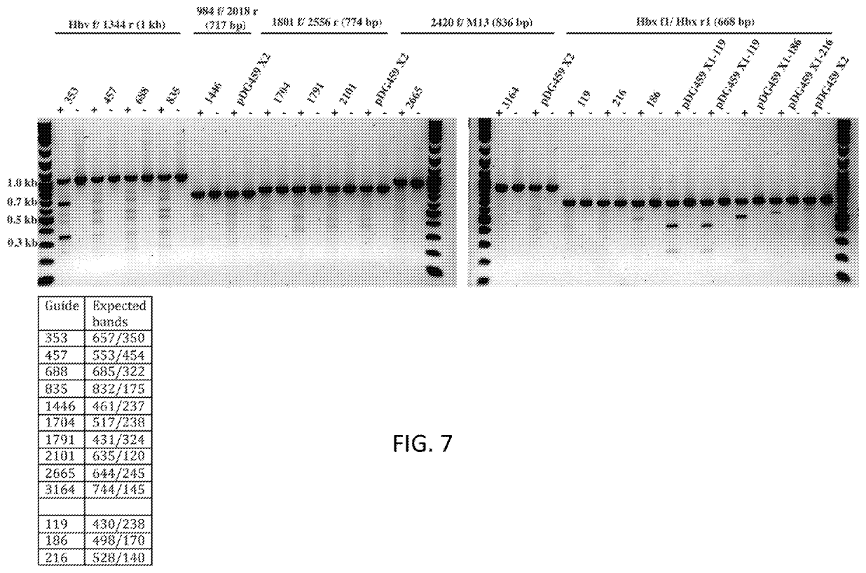

FIG. 7 shows an example of a T7 assay to assess cleavage by sgRNAs. Individual guides were used to determine cleavage of HBV, and assessed by the T7 assay (+/−T7 endonuclease). The pDG459CasX2 plasmid without guides was used as a negative control. Individual guides with CasX2 were cloned into the pBLO plasmid, and individual guides with CasX1 were cloned into the pDG459 plasmid. Guide 353 has the expected bands demonstrating cleavage of the HBV target; guides 1704 & 1791 have faint bands of the expected size; guides 119, 186 and 216 have expected bands. Guide RNA cleavage using the pDG459CasX1 plasmid appears to be more effective than guide RNAs cloned into the pBLOCasX2 plasmid.

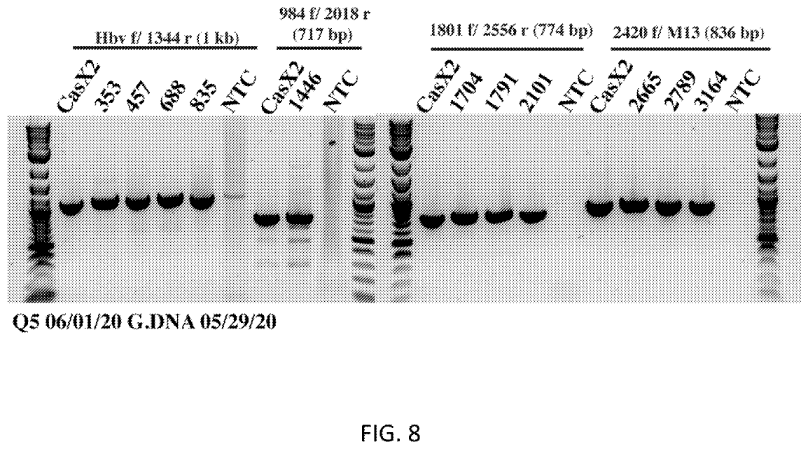

FIG. 8 shows PCR using primers around the expected cut sites which is an assay done prior to the T7 endonuclease assay.

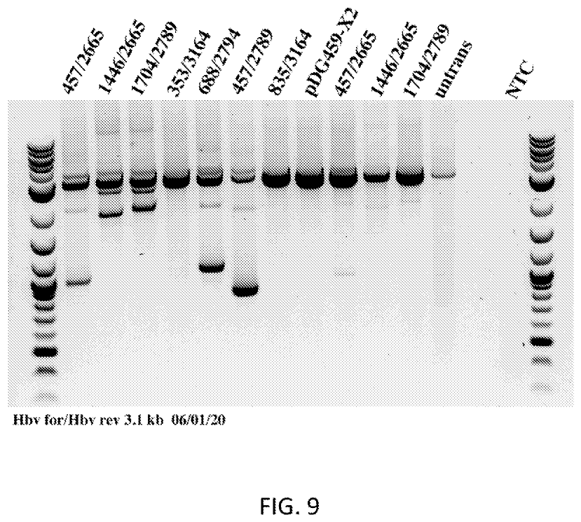

FIG. 9 shows a co-transfection experiment with guides targeting HBV in pDG459-X1 (lanes 3-8) and pDG459-X2 (lanes 10-12). Following co-transfection, cellular DNA was isolated and excision of the HBV plasmid DNA was examined by PCR and agarose gel electrophoresis (shown). Co-transfection with pDG459-X1 (lane 2) and pDG459-X2 (lane 9) plasmids without cloned guide sequences was used as control. Effective cleavage and excision of the intervening DNA sequence of the HBV genome is shown for gRNA pairs 688/2794 and 457/2789 with CasX1.

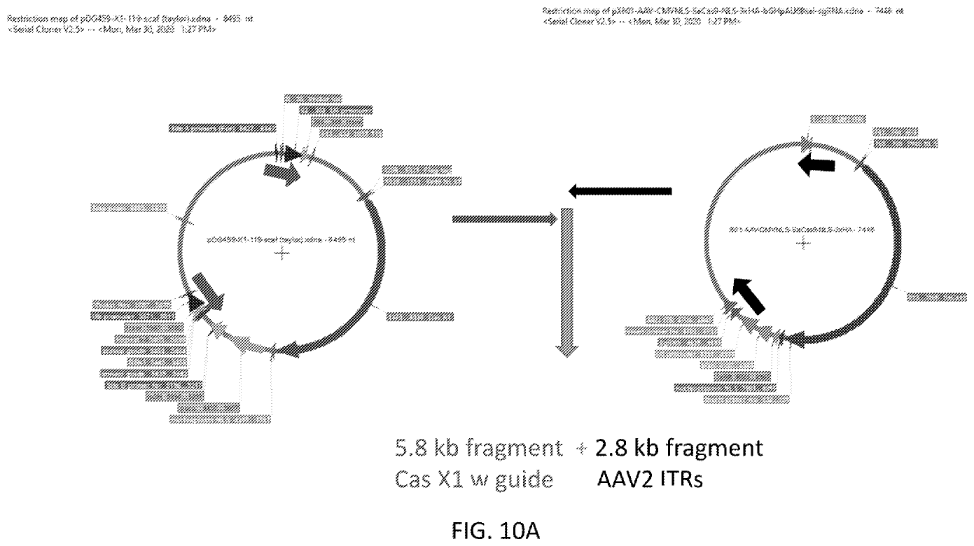

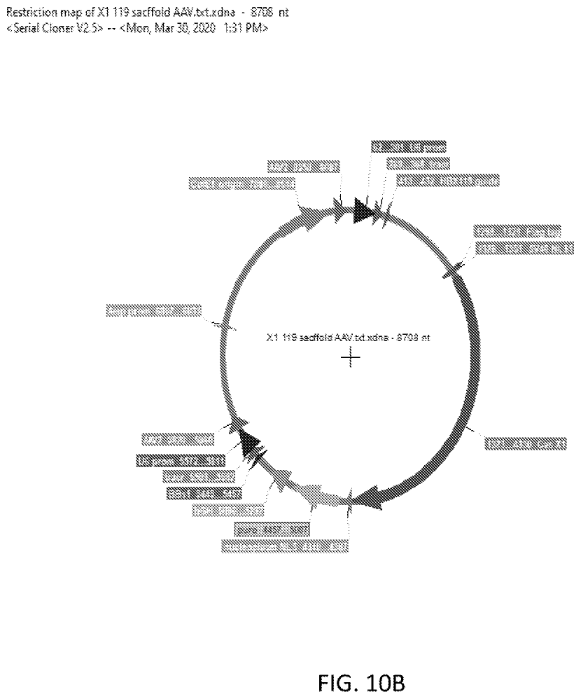

FIG. 10 shows a restriction map for cloning fragments into a vector.

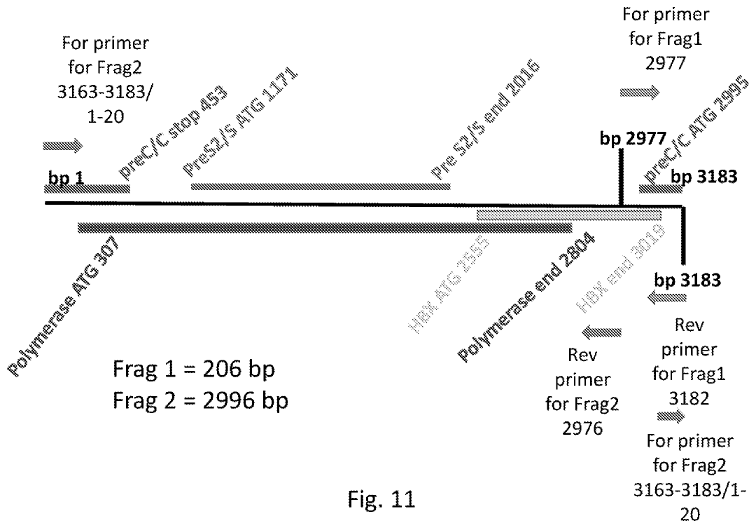

FIG. 11 shows the primers Hbv forward/Hbv reverse for the 3,183 bp in HBV/Topo using the PCR products as a template. FIG. 11 shows primers that can create fragments to be cloned into vectors.

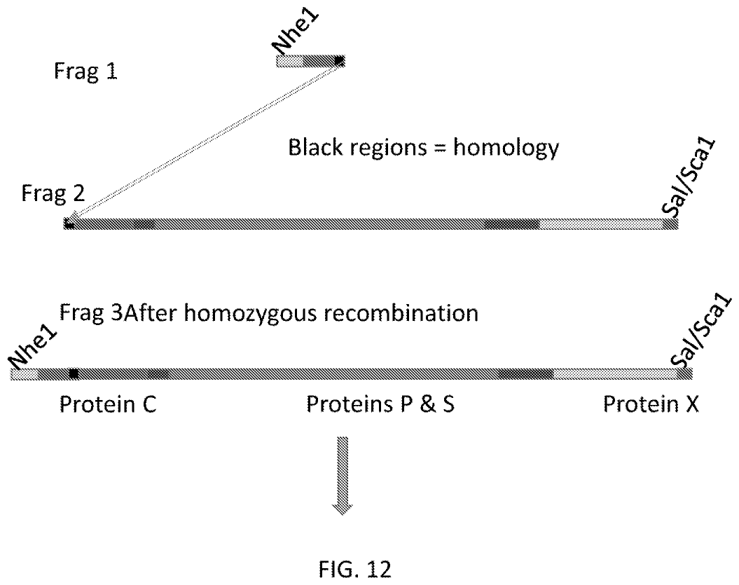

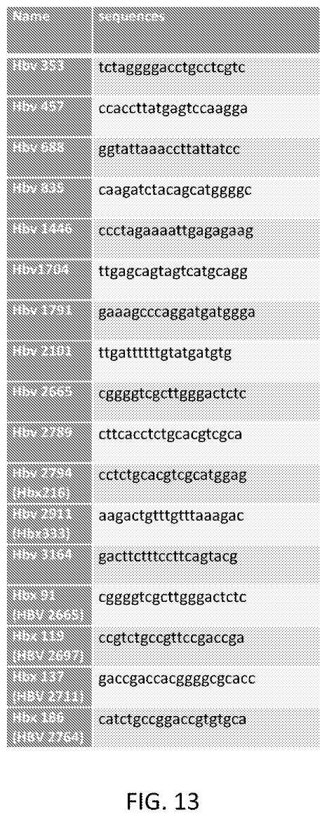

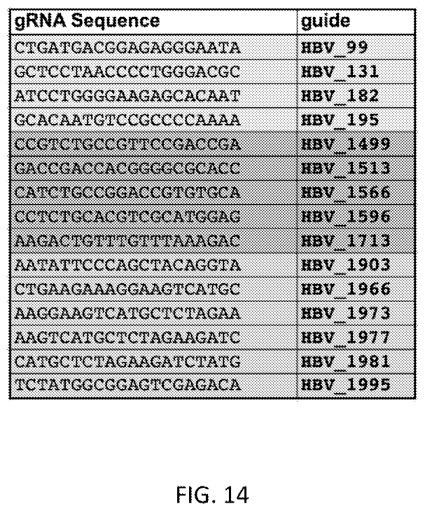

FIG. 12 shows a cloning a strategy. Both ends will have a small region of repeat DNA to ensure each gene is complete (5′ extra HBx DNA; 3′ extra Core DNA). Nhe1 and Sal1 can be used to clone into either pMC.EF1a.MCS.SV40Poly or Nhe1/Sca1 to clone into Lox-Stop-Lox-TOPO.

Those skilled in the art will recognize, or be able to ascertain using no more than routine experimentation, many equivalents to the specific embodiments of the method and compositions described herein. Such equivalents are intended to be encompassed by the following claims.

1. A method of inactivating a virus in a cell comprising administering to a cell comprising a viral genome:
a Cas12d or Cas12e endonuclease, or a nucleic acid construct that encodes the Cas12d or Cas12e endonuclease; and
a first guide RNA (gRNA), or a nucleic acid construct that encodes the first gRNA,
wherein the Cas12d or Cas12e endonuclease cleaves the viral genome at a first target sequence and a second target sequence in the viral genome.

2. The method of claim 1, wherein the first gRNA is complementary to the first target sequence and the second target sequence in the viral genome.

3. The method of claim 1, wherein the first gRNA hybridizes to the first target sequence and the second target sequence in the viral genome resulting in Cas12d or Cas12e endonuclease cleavage at the first target sequence and the second target sequence in the viral genome.

4. The method of claim 1, further comprising a second gRNA.

5. The method of claim 4, wherein the first gRNA is complementary to the first target sequence of the viral genome and the second gRNA is complementary to the second target sequence in the viral genome.

6. The method of claim 5, wherein the first gRNA hybridizes to the first target sequence in the viral genome and the second gRNA hybridizes to the second target sequence in the viral genome resulting in Cas12d or Cas12e endonuclease cleavage at the first target sequence and the second target sequence in the viral genome.

7. The method of claim 1, wherein the cleavage at the first target sequence and the second target sequence results in 5′ single stranded DNA (ssDNA) overhangs at the first target sequence and the second target sequence.

8. The method of claim 7, wherein the 5′ ssDNA overhangs at the first target sequence and the second target sequence have complementary overlapping sequences.

9. The method of claim 8, further comprising hybridizing the 5′ ssDNA overhangs of the first target sequence and the second target sequence.

10. The method of claim 7, wherein the sequence adjacent to the 5′ ssDNA overhangs at the first target sequence and the second target sequence have homologous sequences

11. The method of claim 10, wherein the homologous sequences within or adjacent to the first target sequence and the second target sequence promote microhomology mediated end joining.

12. The method of claim 11, wherein the viral sequence, or some portion of it, between the first target sequence and the second target sequence is removed from the viral or proviral genome.

13. The method of claim 1, wherein the cell comprises a cellular genome and the viral genome is integrated in the cellular genome.

14. The method of claim 1, wherein the viral genome exists as an independent genomic element.

15.-17. (canceled)

18. The method of claim 1, wherein the virus is from the family Retroviridae, Hepadnaviridae, Herpesviridae, Adenoviridae, Papillomaviridae, Poxviridae, Polyomaviridae, Asfarviridae.

19.-21. (canceled)

22. The method of claim 1, wherein the cell's DNA is not cleaved by the Cas12d.

23.-28. (canceled)

29. The method of claim 1, wherein the step of administering to a cell comprises administering to a subject comprising a host cell.

30.-31. (canceled)

32. The method of claim 1, wherein the nucleic acid construct that encodes the Cas12d or Cas12e endonuclease is the same nucleic acid construct that encodes at least one of the first gRNA or second gRNA.

33. (canceled)

34. The method of claim 1, wherein at least one of the first target sequence or second target sequence is a sequence from Table 1 or Table 2.

35.-45. (canceled)

46. A method of treating a subject having a cell comprising a viral genome comprising administering to the subject:
a Cas12d or Cas12e endonuclease, or a nucleic acid construct that encodes the Cas12d or Cas12e endonuclease;
a first gRNA, or a nucleic acid construct that encode the first gRNA, wherein the first gRNA is complementary to a first target sequence in the viral genome of the virus; and
a second gRNA, or a nucleic acid construct that encodes the second gRNA, wherein the second gRNA is complementary to a second target sequence in the viral genome of the virus;
wherein the first gRNA hybridizes to the first target sequence in the viral genome and the second gRNA hybridizes to the second target sequence in the viral genome resulting in CasX or CasY endonuclease cleavage at the first target sequence and the second target sequence in the viral genome.


SEQUENCE LISTING (224 sequences)

SEQ ID NO: 1 - DNA, 24 bp, Hepatitis B virus
  source 1..24: mol_type = genomic DNA
ttccacaaccttccaccaaactct

SEQ ID NO: 2 - DNA, 24 bp, Hepatitis B virus
  source 1..24: mol_type = genomic DNA
ttcctgaactggagccaccagcag

SEQ ID NO: 3 - DNA, 24 bp, Hepatitis B virus
  source 1..24: mol_type = genomic DNA
ttccaccaaactctgcaagatccc

SEQ ID NO: 4 - DNA, 24 bp, Hepatitis B virus
  source 1..24: mol_type = genomic DNA
ttcagcgcagggtccccaatcctc

SEQ ID NO: 5 - DNA, 24 bp, Hepatitis B virus
  source 1..24: mol_type = genomic DNA
ttccctgctggtggctccagttca

SEQ ID NO: 6 - DNA, 24 bp, Hepatitis B virus
  source 1..24: mol_type = genomic DNA
ttctccatgttcagcgcagggtcc

SEQ ID NO: 7 - DNA, 24 bp, Hepatitis B virus
  source 1..24: mol_type = genomic DNA
ttcaggaacagtaaaccctgttct

SEQ ID NO: 8 - DNA, 24 bp, Hepatitis B virus
  source 1..24: mol_type = genomic DNA
ttcttgtcaacaagaaaaaccccg

SEQ ID NO: 9 - DNA, 24 bp, Hepatitis B virus
  source 1..24: mol_type = genomic DNA
ttctgactactgcctctcccttat

SEQ ID NO: 10 - DNA, 24 bp, Hepatitis B virus
  source 1..24: mol_type = genomic DNA
ttccccctagaaaattgagagaag

SEQ ID NO: 11 - DNA, 24 bp, Hepatitis B virus
  source 1..24: mol_type = genomic DNA
ttctcgaggattggggaccctgcg

SEQ ID NO: 12 - DNA, 24 bp, Hepatitis B virus
  source 1..24: mol_type = genomic DNA
ttccttgagcagtagtcatgcagg

SEQ ID NO: 13 - DNA, 24 bp, Hepatitis B virus
  source 1..24: mol_type = genomic DNA
ttcctaggaccccttctcgtgtta

SEQ ID NO: 14 - DNA, 24 bp, Hepatitis B virus
  source 1..24: mol_type = genomic DNA
ttccgtccgaaggtttggtacagc

SEQ ID NO: 15 - DNA, 24 bp, Hepatitis B virus
  source 1..24: mol_type = genomic DNA
ttctcgtgttacaggcggggtttt

SEQ ID NO: 16 - DNA, 24 bp, Hepatitis B virus
  source 1..24: mol_type = genomic DNA
ttccgaaagcccaggatgatggga

SEQ ID NO: 17 - DNA, 24 bp, Hepatitis B virus
  source 1..24: mol_type = genomic DNA
ttcttgttgacaagaatcctcaca

SEQ ID NO: 18 - DNA, 24 bp, Hepatitis B virus
  source 1..24: mol_type = genomic DNA
ttcttgtggcaaggacccataaca

SEQ ID NO: 19 - DNA, 24 bp, Hepatitis B virus
  source 1..24: mol_type = genomic DNA
ttctctcaattttctagggggaac

SEQ ID NO: 20 - DNA, 24 bp, Hepatitis B virus
  source 1..24: mol_type = genomic DNA
ttctttgattttttgtatgatgtg

SEQ ID NO: 21 - DNA, 24 bp, Hepatitis B virus
  source 1..24: mol_type = genomic DNA
ttctagggggaactaccgtgtgtc

SEQ ID NO: 22 - DNA, 24 bp, Hepatitis B virus
  source 1..24: mol_type = genomic DNA
ttctaaaacattctttgatttttt

SEQ ID NO: 23 - DNA, 24 bp, Hepatitis B virus
  source 1..24: mol_type = genomic DNA
ttcgcagtccccaacctccaatca

SEQ ID NO: 24 - DNA, 24 bp, Hepatitis B virus
  source 1..24: mol_type = genomic DNA
ttccaatcaataggcctgttaata

SEQ ID NO: 25 - DNA, 24 bp, Hepatitis B virus
  source 1..24: mol_type = genomic DNA
ttcctcttcatcctgctgctatgc

SEQ ID NO: 26 - DNA, 24 bp, Hepatitis B virus
  source 1..24: mol_type = genomic DNA
ttcgttgacatactttccaatcaa

SEQ ID NO: 27 - DNA, 24 bp, Hepatitis B virus
  source 1..24: mol_type = genomic DNA
ttcatcctgctgctatgcctcatc

SEQ ID NO: 28 - DNA, 24 bp, Hepatitis B virus
  source 1..24: mol_type = genomic DNA
ttcaggtattgtttacacagaaag

SEQ ID NO: 29 - DNA, 24 bp, Hepatitis B virus
  source 1..24: mol_type = genomic DNA
ttcttgttggttcttctggactat

SEQ ID NO: 30 - DNA, 24 bp, Hepatitis B virus
  source 1..24: mol_type = genomic DNA
ttccacgcatgcgctgatggccca

SEQ ID NO: 31 - DNA, 24 bp, Hepatitis B virus
  source 1..24: mol_type = genomic DNA
ttcttctggactatcaaggtatgt

SEQ ID NO: 32 - DNA, 24 bp, Hepatitis B virus
  source 1..24: mol_type = genomic DNA
ttccgcagtatggatcggcagagg

SEQ ID NO: 33 - DNA, 24 bp, Hepatitis B virus
  source 1..24: mol_type = genomic DNA
ttctggactatcaaggtatgttgc

SEQ ID NO: 34 - DNA, 24 bp, Hepatitis B virus
  source 1..24: mol_type = genomic DNA
ttcagcgccgacgggacgtaaaca

SEQ ID NO: 35 - DNA, 24 bp, Hepatitis B virus
  source 1..24: mol_type = genomic DNA
ttccaggatcctcaacaaccagca

SEQ ID NO: 36 - DNA, 24 bp, Hepatitis B virus
  source 1..24: mol_type = genomic DNA
ttcacggtggtctccatgcgacgt

SEQ ID NO: 37 - DNA, 24 bp, Hepatitis B virus
  source 1..24: mol_type = genomic DNA
ttcggacggaaattgcacctgtat

SEQ ID NO: 38 - DNA, 24 bp, Hepatitis B virus
  source 1..24: mol_type = genomic DNA
ttctttataagggtcgatgtccat

SEQ ID NO: 39 - DNA, 24 bp, Hepatitis B virus
  source 1..24: mol_type = genomic DNA
ttcccatcccatcatcctgggctt

SEQ ID NO: 40 - DNA, 24 bp, Hepatitis B virus
  source 1..24: mol_type = genomic DNA
ttcccgatacagagctgaggcggt

SEQ ID NO: 41 - DNA, 24 bp, Hepatitis B virus
  source 1..24: mol_type = genomic DNA
ttcggaaaattcctatgggagtgg

SEQ ID NO: 42 - DNA, 24 bp, Hepatitis B virus
  source 1..24: mol_type = genomic DNA
ttccccccagcaaagaattgcttg

SEQ ID NO: 43 - DNA, 24 bp, Hepatitis B virus
  source 1..24: mol_type = genomic DNA
ttcctatgggagtgggcctcagcc

SEQ ID NO: 44 - DNA, 24 bp, Hepatitis B virus
  source 1..24: mol_type = genomic DNA
ttccaaattaacacccacccaggt

SEQ ID NO: 45 - DNA, 24 bp, Hepatitis B virus
  source 1..24: mol_type = genomic DNA
ttctcctggctcagtttactagtg

SEQ ID NO: 46 - DNA, 24 bp, Hepatitis B virus
  source 1..24: mol_type = genomic DNA
ttccaaaagtgagacaagaaatgt

SEQ ID NO: 47 - DNA, 24 bp, Hepatitis B virus
  source 1..24: mol_type = genomic DNA
ttcagtggttcgtagggctttccc

SEQ ID NO: 48 - DNA, 24 bp, Hepatitis B virus
  source 1..24: mol_type = genomic DNA
ttctcttccaaaagtgagacaaga

SEQ ID NO: 49 - DNA, 24 bp, Hepatitis B virus
  source 1..24: mol_type = genomic DNA
ttcgtagggctttcccccactgtt

SEQ ID NO: 50 - DNA, 24 bp, Hepatitis B virus
  source 1..24: mol_type = genomic DNA
ttctaggggacctgcctcgtcgtc

SEQ ID NO: 51 - DNA, 24 bp, Hepatitis B virus
  source 1..24: mol_type = genomic DNA
ttcccccactgtttggctttcagt

SEQ ID NO: 52 - DNA, 24 bp, Hepatitis B virus
  source 1..24: mol_type = genomic DNA
ttcttctaggggacctgcctcgtc

SEQ ID NO: 53 - DNA, 24 bp, Hepatitis B virus
  source 1..24: mol_type = genomic DNA
ttcagttatatggatgatgtggta

SEQ ID NO: 54 - DNA, 24 bp, Hepatitis B virus
  source 1..24: mol_type = genomic DNA
ttcttcttctaggggacctgcctc

SEQ ID NO: 55 - DNA, 24 bp, Hepatitis B virus
  source 1..24: mol_type = genomic DNA
ttcttttgtctttgggtatacatt

SEQ ID NO: 56 - DNA, 24 bp, Hepatitis B virus
  source 1..24: mol_type = genomic DNA
ttcgtctgcgaggcgagggagttc

SEQ ID NO: 57 - DNA, 24 bp, Hepatitis B virus
  source 1..24: mol_type = genomic DNA
ttcctattaacaggcctattgatt

SEQ ID NO: 58 - DNA, 24 bp, Hepatitis B virus
  source 1..24: mol_type = genomic DNA
ttctgcgacgcggcgattgagacc

SEQ ID NO: 59 - DNA, 24 bp, Hepatitis B virus
  source 1..24: mol_type = genomic DNA
ttcaatctaagcaggctttcactt

SEQ ID NO: 60 - DNA, 24 bp, Hepatitis B virus
  source 1..24: mol_type = genomic DNA
ttcccgagattgagatcttctgcg

SEQ ID NO: 61 - DNA, 24 bp, Hepatitis B virus
  source 1..24: mol_type = genomic DNA
ttcactttctcgccaacttacaag

SEQ ID NO: 62 - DNA, 24 bp, Hepatitis B virus
  source 1..24: mol_type = genomic DNA
ttccccaccttatgagtccaagga

SEQ ID NO: 63 - DNA, 24 bp, Hepatitis B virus
  source 1..24: mol_type = genomic DNA
ttctcgccaacttacaaggccttt

SEQ ID NO: 64 - DNA, 24 bp, Hepatitis B virus
  source 1..24: mol_type = genomic DNA
ttccaatgaggattaaagacaggt

SEQ ID NO: 65 - DNA, 24 bp, Hepatitis B virus
  source 1..24: mol_type = genomic DNA
ttctgtgtaaacaatacctgaacc

SEQ ID NO: 66 - DNA, 24 bp, Hepatitis B virus
  source 1..24: mol_type = genomic DNA
ttcacattttttgataatgtcttg

SEQ ID NO: 67 - DNA, 24 bp, Hepatitis B virus
  source 1..24: mol_type = genomic DNA
ttcggctcctctgccgatccatac

SEQ ID NO: 68 - DNA, 24 bp, Hepatitis B virus
  source 1..24: mol_type = genomic DNA
ttctcattaactgtgagtgggcct

SEQ ID NO: 69 - DNA, 24 bp, Hepatitis B virus
  source 1..24: mol_type = genomic DNA
ttccatggctgctaggctgtgctg

SEQ ID NO: 70 - DNA, 24 bp, Hepatitis B virus
  source 1..24: mol_type = genomic DNA
ttcttttctcattaactgtgagtg

SEQ ID NO: 71 - DNA, 24 bp, Hepatitis B virus
  source 1..24: mol_type = genomic DNA
ttctcggggtcgcttgggactctc

SEQ ID NO: 72 - DNA, 24 bp, Hepatitis B virus
  source 1..24: mol_type = genomic DNA
ttctggataataaggtttaatacc

SEQ ID NO: 73 - DNA, 24 bp, Hepatitis B virus
  source 1..24: mol_type = genomic DNA
ttctccgtctgccgttccgaccga

SEQ ID NO: 74 - DNA, 24 bp, Hepatitis B virus
  source 1..24: mol_type = genomic DNA
ttccatagagtgtgtaaatagtgt

SEQ ID NO: 75 - DNA, 24 bp, Hepatitis B virus
  source 1..24: mol_type = genomic DNA
ttccgaccgaccacggggcgcacc

SEQ ID NO: 76 - DNA, 24 bp, Hepatitis B virus
  source 1..24: mol_type = genomic DNA
ttctctcttatataatatacccgc

SEQ ID NO: 77 - DNA, 24 bp, Hepatitis B virus
  source 1..24: mol_type = genomic DNA
ttctcatctgccggaccgtgtgca

SEQ ID NO: 78 - DNA, 24 bp, Hepatitis B virus
  source 1..24: mol_type = genomic DNA
ttcccaagaatatggtgacccaca

SEQ ID NO: 79 - DNA, 24 bp, Hepatitis B virus
  source 1..24: mol_type = genomic DNA
ttcgcttcacctctgcacgtcgca

SEQ ID NO: 80 - DNA, 24 bp, Hepatitis B virus
  source 1..24: mol_type = genomic DNA
ttctgccccatgctgtagatcttg

SEQ ID NO: 81 - DNA, 24 bp, Hepatitis B virus
  source 1..24: mol_type = genomic DNA
ttcacctctgcacgtcgcatggag

SEQ ID NO: 82 - DNA, 24 bp, Hepatitis B virus
  source 1..24: mol_type = genomic DNA
ttcctgactggcgattggtggagg

SEQ ID NO: 83 - DNA, 24 bp, Hepatitis B virus
  source 1..24: mol_type = genomic DNA
ttcaaagactgtttgtttaaagac

SEQ ID NO: 84 - DNA, 24 bp, Hepatitis B virus
  source 1..24: mol_type = genomic DNA
ttctcaaaggtggagacagcgggg

SEQ ID NO: 85 - DNA, 24 bp, Hepatitis B virus
  source 1..24: mol_type = genomic DNA
ttcacctctgcctaatcatctctt

SEQ ID NO: 86 - DNA, 24 bp, Hepatitis B virus
  source 1..24: mol_type = genomic DNA
ttcatgtcctactgttcaagcctc

SEQ ID NO: 87 - DNA, 24 bp, Hepatitis B virus
  source 1..24: mol_type = genomic DNA
ttcaagcctccaagctgtgccttg

SEQ ID NO: 88 - DNA, 24 bp, Hepatitis B virus
  source 1..24: mol_type = genomic DNA
ttctgacttctttccttcagtacg

SEQ ID NO: 89 - DNA, 24 bp, Hepatitis B virus
  source 1..24: mol_type = genomic DNA
ttctttccttcagtacgagatctt

SEQ ID NO: 90 - DNA, 24 bp, Hepatitis B virus
  source 1..24: mol_type = genomic DNA
ttccttcagtacgagatcttctag

SEQ ID NO: 91 - DNA, 24 bp, Hepatitis B virus
  source 1..24: mol_type = genomic DNA
ttcagtacgagatcttctagatac

SEQ ID NO: 92 - DNA, 24 bp, Hepatitis B virus
  source 1..24: mol_type = genomic DNA
ttctagataccgcctcagctctgt

SEQ ID NO: 93 - DNA, 24 bp, Hepatitis B virus
  source 1..24: mol_type = genomic DNA
ttcacctcaccatactgcactcag

SEQ ID NO: 94 - DNA, 24 bp, Hepatitis B virus
  source 1..24: mol_type = genomic DNA
ttctttgctggggggaactaatga

SEQ ID NO: 95 - DNA, 24 bp, Hepatitis B virus
  source 1..24: mol_type = genomic DNA
ttcaggcaactcttgtggtttcac

SEQ ID NO: 96 - DNA, 24 bp, Hepatitis B virus
  source 1..24: mol_type = genomic DNA
ttcacatttcttgtctcacttttg

SEQ ID NO: 97 - DNA, 24 bp, Hepatitis B virus
  source 1..24: mol_type = genomic DNA
ttcttgtctcacttttggaagaga

SEQ ID NO: 98 - DNA, 24 bp, Hepatitis B virus
  source 1..24: mol_type = genomic DNA
ttcggagtgtggattcgcactcct

SEQ ID NO: 99 - DNA, 24 bp, Hepatitis B virus
  source 1..24: mol_type = genomic DNA
ttcgcactcctccagcttatagac

SEQ ID NO: 100 - DNA, 24 bp, Hepatitis B virus
  source 1..24: mol_type = genomic DNA
ttccggagactactgttgttagac

SEQ ID NO: 101 - DNA, 24 bp, Hepatitis B virus
  source 1..24: mol_type = genomic DNA
ttccttggactcataaggtgggga

SEQ ID NO: 102 - DNA, 24 bp, Hepatitis B virus
  source 1..24: mol_type = genomic DNA
ttcttctactgtacctgtctttaa

SEQ ID NO: 103 - DNA, 24 bp, Hepatitis B virus
  source 1..24: mol_type = genomic DNA
ttctactgtacctgtctttaatcc

SEQ ID NO: 104 - DNA, 24 bp, Hepatitis B virus
  source 1..24: mol_type = genomic DNA
ttcctaatatacatttacaccaag

SEQ ID NO: 105 - DNA, 24 bp, Hepatitis B virus
  source 1..24: mol_type = genomic DNA
ttccaaactagacactatttacac

SEQ ID NO: 106 - DNA, 24 bp, Hepatitis B virus
  source 1..24: mol_type = genomic DNA
ttcttgggaacaagatctacagca

SEQ ID NO: 107 - DNA, 24 bp, Hepatitis B virus
  source 1..24: mol_type = genomic DNA
ttccaccagcaatcctctgggatt

SEQ ID NO: 108 - DNA, 24 bp, Hepatitis B virus
  source 1..24: mol_type = other DNA
ttctttcccgaccaccagttggat

SEQ ID NO: 109 - DNA, 24 bp, Hepatitis B virus
  source 1..24: mol_type = genomic DNA
ttcccgaccaccagttggatccag

SEQ ID NO: 110 - DNA, 24 bp, Hepatitis B virus
  source 1..24: mol_type = genomic DNA
ttcagagcaaacaccgcaaatcca

SEQ ID NO: 111 - DNA, 24 bp, Hepatitis B virus
  source 1..24: mol_type = genomic DNA
ttcaatcccaacaaggacacctgg

SEQ ID NO: 112 - DNA, 24 bp, Hepatitis B virus
  source 1..24: mol_type = genomic DNA
ttcgggctgggtttcaccccaccg

SEQ ID NO: 113 - DNA, 24 bp, Hepatitis B virus
  source 1..24: mol_type = genomic DNA
ttcaccccaccgcacggaggcctt

SEQ ID NO: 114 - DNA, 20 bp, synthetic construct
  misc_feature 1..20: note = synthetic construct; sgRNA
  source 1..20: mol_type = other DNA
tctaggggacctgcctcgtc

SEQ ID NO: 115 - DNA, 20 bp, synthetic construct
  misc_feature 1..20: note = synthetic construct; sgRNA
  source 1..20: mol_type = other DNA
gagctactgtggagttactc

SEQ ID NO: 116 - DNA, 20 bp, synthetic construct
  misc_feature 1..20: note = synthetic construct; sgRNA
  source 1..20: mol_type = other DNA
ccaccttatgagtccaagga

SEQ ID NO: 117 - DNA, 20 bp, synthetic construct
  misc_feature 1..20: note = synthetic construct; sgRNA
  source 1..20: mol_type = other DNA
tcctgctgctatgcctcatc

SEQ ID NO: 118 - DNA, 20 bp, synthetic construct
  misc_feature 1..20: note = synthetic construct; sgRNA
  source 1..20: mol_type = other DNA
cattttttgataatgtcttg

SEQ ID NO: 119 - DNA, 20 bp, synthetic construct
  misc_feature 1..20: note = synthetic construct; sgRNA
  source 1..20: mol_type = other DNA
gttatatggatgatgtggta

SEQ ID NO: 120 - DNA, 20 bp, synthetic construct
  misc_feature 1..20: note = synthetic construct; sgRNA
  source 1..20: mol_type = other DNA
cattaactgtgagtgggcct

SEQ ID NO: 121 - DNA, 20 bp, synthetic construct
  misc_feature 1..20: note = synthetic construct; sgRNA
  source 1..20: mol_type = other DNA
gtggttcgtagggctttccc

SEQ ID NO: 122 - DNA, 20 bp, synthetic construct
  misc_feature 1..20: note = synthetic construct; sgRNA
  source 1..20: mol_type = other DNA
atagagtgtgtaaatagtgt

SEQ ID NO: 123 - DNA, 20 bp, synthetic construct
  misc_feature 1..20: note = synthetic construct; sgRNA
  source 1..20: mol_type = other DNA
gttatatggatgatgtggta

SEQ ID NO: 124 - DNA, 20 bp, synthetic construct
  misc_feature 1..20: note = synthetic construct; sgRNA
  source 1..20: mol_type = other DNA
ctcttatataatatacccgc

SEQ ID NO: 125 - DNA, 20 bp, synthetic construct
  misc_feature 1..20: note = synthetic construct; sgRNA
  source 1..20: mol_type = other DNA
tgttggttcttctggactat

SEQ ID NO: 126 - DNA, 20 bp, synthetic construct
  misc_feature 1..20: note = synthetic construct; sgRNA
  source 1..20: mol_type = other DNA
ctcttatataatatacccgc

SEQ ID NO: 127 - DNA, 20 bp, synthetic construct
  misc_feature 1..20: note = synthetic construct; sgRNA
  source 1..20: mol_type = other DNA
gttatatggatgatgtggta

SEQ ID NO: 128 - DNA, 20 bp, synthetic construct
  misc_feature 1..20: note = synthetic construct; sgRNA
  source 1..20: mol_type = other DNA
gccccatgctgtagatcttg

SEQ ID NO: 129 - DNA, 20 bp, synthetic construct
  misc_feature 1..20: note = synthetic construct; sgRNA
  source 1..20: mol_type = other DNA
gacttctttccttcagtacg

SEQ ID NO: 130 - DNA, 20 bp, synthetic construct
  misc_feature 1..20: note = synthetic construct; sgRNA
  source 1..20: mol_type = other DNA
actgcatggcctgaggatga

SEQ ID NO: 131 - DNA, 20 bp, synthetic construct
  misc_feature 1..20: note = synthetic construct; sgRNA
  source 1..20: mol_type = other DNA
tcttcatcctgctgctatgc

SEQ ID NO: 132 - DNA, 20 bp, synthetic construct
  misc_feature 1..20: note = synthetic construct; sgRNA
  source 1..20: mol_type = other DNA
actgcatggcctgaggatga

SEQ ID NO: 133 - DNA, 20 bp, synthetic construct
  misc_feature 1..20: note = synthetic construct; sgRNA
  source 1..20: mol_type = other DNA
gaaaattcctatgggagtgg

SEQ ID NO: 134 - DNA, 19 bp, Human immunodeficiency virus
  source 1..19: mol_type = genomic DNA
gctaatcagggaagtagcc

SEQ ID NO: 135 - DNA, 20 bp, Human immunodeficiency virus
  source 1..20: mol_type = genomic DNA
gcattatcagaaggagccac

SEQ ID NO: 136 - DNA, 20 bp, Hepatitis B virus
  source 1..20: mol_type = genomic DNA
ataagattgacgatatggca

SEQ ID NO: 137 - DNA, 21 bp, Hepatitis B virus
  source 1..21: mol_type = genomic DNA
catctgccggaccgtgtgcac

SEQ ID NO: 138 - DNA, 20 bp, Human T-cell lymphotropic virus
  source 1..20: mol_type = genomic DNA
gggatagtgggctttaggcg

SEQ ID NO: 139 - DNA, 20 bp, Human T-cell lymphotropic virus
  source 1..20: mol_type = genomic DNA
ttaataccgaacccagccaa

SEQ ID NO: 140 - DNA, 20 bp, synthetic construct
  misc_feature 1..20: note = synthetic construct; JCV sequence
  source 1..20: mol_type = other DNA
tttctggtgggatcaggaac

SEQ ID NO: 141 - DNA, 20 bp, synthetic construct
  misc_feature 1..20: note = synthetic construct; JCV sequence
  source 1..20: mol_type = other DNA
gataagcttttctcatgaca

SEQ ID NO: 142 - DNA, 20 bp, Hepatitis B virus
  source 1..20: mol_type = genomic DNA
tctaggggacctgcctcgtc

SEQ ID NO: 143 - DNA, 20 bp, Hepatitis B virus
  source 1..20: mol_type = genomic DNA
ccaccttatgagtccaagga

SEQ ID NO: 144 - DNA, 20 bp, Hepatitis B virus
  source 1..20: mol_type = genomic DNA
ggtattaaaccttattatcc

SEQ ID NO: 145 - DNA, 20 bp, Hepatitis B virus
  source 1..20: mol_type = genomic DNA
ccctagaaaattgagagaag

SEQ ID NO: 146 - DNA, 20 bp, Hepatitis B virus
  source 1..20: mol_type = genomic DNA
ttgagcagtagtcatgcagg

SEQ ID NO: 147 - DNA, 20 bp, Hepatitis B virus
  source 1..20: mol_type = genomic DNA
gaaagcccaggatgatggga

SEQ ID NO: 148 - DNA, 20 bp, Hepatitis B virus
  source 1..20: mol_type = genomic DNA
ttgattttttgtatgatgtg

SEQ ID NO: 149 - DNA, 20 bp, Hepatitis B virus
  source 1..20: mol_type = genomic DNA
cggggtcgcttgggactctc

SEQ ID NO: 150 - DNA, 20 bp, Hepatitis B virus
  source 1..20: mol_type = genomic DNA
cttcacctctgcacgtcgca

SEQ ID NO: 151 - DNA, 20 bp, Hepatitis B virus
  source 1..20: mol_type = genomic DNA
cctctgcacgtcgcatggag

SEQ ID NO: 152 - DNA, 20 bp, Hepatitis B virus
  source 1..20: mol_type = genomic DNA
aagactgtttgtttaaagac

SEQ ID NO: 153 - DNA, 20 bp, Hepatitis B virus
  source 1..20: mol_type = genomic DNA
gacttctttccttcagtacg

SEQ ID NO: 154 - DNA, 20 bp, Hepatitis B virus
  source 1..20: mol_type = genomic DNA
cggggtcgcttgggactctc

SEQ ID NO: 155 - DNA, 20 bp, Hepatitis B virus
  source 1..20: mol_type = genomic DNA
ccgtctgccgttccgaccga

SEQ ID NO: 156 - DNA, 20 bp, Hepatitis B virus
  source 1..20: mol_type = genomic DNA
gaccgaccacggggcgcacc

SEQ ID NO: 157 - DNA, 20 bp, Hepatitis B virus
  source 1..20: mol_type = genomic DNA
catctgccggaccgtgtgca

SEQ ID NO: 158 - DNA, 20 bp, Hepatitis B virus
  source 1..20: mol_type = genomic DNA
ctgatgacggagagggaata

SEQ ID NO: 159 - DNA, 20 bp, Hepatitis B virus
  source 1..20: mol_type = genomic DNA
gctcctaacccctgggacgc

SEQ ID NO: 160 - DNA, 20 bp, Hepatitis B virus
  source 1..20: mol_type = genomic DNA
atcctggggaagagcacaat

SEQ ID NO: 161 - DNA, 20 bp, Hepatitis B virus
  source 1..20: mol_type = genomic DNA
gcacaatgtccgccccaaaa

SEQ ID NO: 162 - DNA, 20 bp, Hepatitis B virus
  source 1..20: mol_type = genomic DNA
ccgtctgccgttccgaccga

SEQ ID NO: 163 - DNA, 20 bp, Hepatitis B virus
  source 1..20: mol_type = genomic DNA
gaccgaccacggggcgcacc

SEQ ID NO: 164 - DNA, 20 bp, Hepatitis B virus
  source 1..20: mol_type = genomic DNA
catctgccggaccgtgtgca

SEQ ID NO: 165 - DNA, 20 bp, Hepatitis B virus
  source 1..20: mol_type = genomic DNA
cctctgcacgtcgcatggag

SEQ ID NO: 166 - DNA, 20 bp, Hepatitis B virus
  source 1..20: mol_type = genomic DNA
aagactgtttgtttaaagac

SEQ ID NO: 167 - DNA, 20 bp, Hepatitis B virus
  source 1..20: mol_type = genomic DNA
aatattcccagctacaggta

SEQ ID NO: 168 - DNA, 20 bp, Hepatitis B virus
  source 1..20: mol_type = genomic DNA
ctgaagaaaggaagtcatgc

SEQ ID NO: 169 - DNA, 20 bp, Hepatitis B virus
  source 1..20: mol_type = genomic DNA
aaggaagtcatgctctagaa

SEQ ID NO: 170 - DNA, 20 bp, Hepatitis B virus
  source 1..20: mol_type = genomic DNA
aagtcatgctctagaagatc

SEQ ID NO: 171 - DNA, 20 bp, Hepatitis B virus
  source 1..20: mol_type = genomic DNA
catgctctagaagatctatg

SEQ ID NO: 172 - DNA, 20 bp, Hepatitis B virus
  source 1..20: mol_type = genomic DNA
tctatggcggagtcgagaca

SEQ ID NO: 173 - DNA, 77 bp, synthetic construct
  misc_feature 1..77: note = synthetic construct; sgRNA
  source 1..77: mol_type = other DNA
gtccccttctccgtctgccgttccgaccgaccacggggcgcacctctcttttacgcggactccccgtctgtgccttc

SEQ ID NO: 174 - DNA, 76 bp, synthetic construct
  misc_feature 1..76: note = synthetic construct; sgRNA
  source 1..76: mol_type = other DNA
caggggaagaggcagacggcaaggctggctggtgccccgcgtggagagaaatgcgcctgaggggcagacacggaag

SEQ ID NO: 175 - DNA, 51 bp, synthetic construct
  misc_feature 1..51: note = synthetic construct; sgRNA
  source 1..51: mol_type = other DNA
cccttctccgtctgccgttccgaccgaccacggggcgcacctctctcttta

SEQ ID NO: 176 - DNA, 49 bp, synthetic construct
  misc_feature 1..49: note = synthetic construct; sgRNA
  source 1..49: mol_type = other DNA
gggaagaggcagacggcaaggctggctggtgccccgcgtggagagaaat

SEQ ID NO: 177 - DNA, 40 bp, synthetic construct
  misc_feature 1..40: note = synthetic construct; sgRNA target site
  source 1..40: mol_type = other DNA
acgggaccatgccggacctgcatgactactgctcaaggaa

SEQ ID NO: 178 - DNA, 40 bp, synthetic construct
  misc_feature 1..40: note = synthetic construct; sgRNA target site
  source 1..40: mol_type = other DNA
tgccctggtacggcctggacgtactgatgacgagttcctt

SEQ ID NO: 179 - DNA, 42 bp, synthetic construct
  misc_feature 1..42: note = synthetic construct; sgRNA target site
  source 1..42: mol_type = other DNA
ttcgcttcacctctgcacgtcgcatggagaccaccgtgaacg

SEQ ID NO: 180 - DNA, 42 bp, synthetic construct
  misc_feature 1..42: note = synthetic construct; sgRNA target site
  source 1..42: mol_type = other DNA
aagcgaagtggagacgtgcagcgtacctctggtggcacttgc

SEQ ID NO: 181 - DNA, 29 bp, synthetic construct
  misc_feature 1..29: note = synthetic construct; sgRNA
  source 1..29: mol_type = other DNA
catgccggacctgcatgactactgctcaa

SEQ ID NO: 182 - DNA, 27 bp, synthetic construct
  misc_feature 1..27: note = synthetic construct; sgRNA
  source 1..27: mol_type = other DNA
cttcacctctgcacgtcgcatggagac

SEQ ID NO: 183 - DNA, 18 bp, synthetic construct
  misc_feature 1..18: note = synthetic construct; Hbv target site
  source 1..18: mol_type = other DNA
catgccggacatggagac

SEQ ID NO: 184 - DNA, 29 bp, synthetic construct
  misc_feature 1..29: note = synthetic construct; sgRNA
  source 1..29: mol_type = other DNA
catgccggacctgcatgactactgctcaa

SEQ ID NO: 185 - DNA, 27 bp, synthetic construct
  misc_feature 1..27: note = synthetic construct; sgRNA
  source 1..27: mol_type = other DNA
cttcacctctgcacgtcgcatggagac

SEQ ID NO: 186 - DNA, 18 bp, synthetic construct
  misc_feature 1..18: note = synthetic construct; hbv target site
  source 1..18: mol_type = other DNA
catgccggacctggagac

SEQ ID NO: 187 - DNA, 29 bp, synthetic construct
  misc_feature 1..29: note = synthetic construct; sgRNA
  source 1..29: mol_type = other DNA
catgccggacctgcatgactactgctcaa

SEQ ID NO: 188 - DNA, 27 bp, synthetic construct
  misc_feature 1..27: note = synthetic construct; sgRNA
  source 1..27: mol_type = other DNA
cttcacctctgcacgtcgcatggagac

SEQ ID NO: 189 - DNA, 12 bp, synthetic construct
  misc_feature 1..12: note = synthetic construct; hbv target site
  source 1..12: mol_type = other DNA
catgctggagac

SEQ ID NO: 190 - DNA, 29 bp, synthetic construct
  misc_feature 1..29: note = synthetic construct; sgRNA
  source 1..29: mol_type = other DNA
catgccggacctgcatgactactgctcaa

SEQ ID NO: 191 - DNA, 27 bp, synthetic construct
  misc_feature 1..27: note = synthetic construct; sgRNA
  source 1..27: mol_type = other DNA
cttcacctctgcacgtcgcatggagac

SEQ ID NO: 192 - DNA, 16 bp, synthetic construct
  misc_feature 1..16: note = synthetic construct; hbv target site
  source 1..16: mol_type = other DNA
catgccgcatggagac

SEQ ID NO: 193 - DNA, 20 bp, synthetic construct
  misc_feature 1..20: note = synthetic construct; sgRNA
  source 1..20: mol_type = other DNA
tctaggggacctgcctcgtc

SEQ ID NO: 194 - DNA, 20 bp, synthetic construct
  misc_feature 1..20: note = synthetic construct; sgRNA
  source 1..20: mol_type = other DNA
ccaccttatgagtccaagga

SEQ ID NO: 195 - DNA, 20 bp, synthetic construct
  misc_feature 1..20: note = synthetic construct; sgRNA
  source 1..20: mol_type = other DNA
ggtattaaaccttattatcc

SEQ ID NO: 196 - DNA, 20 bp, synthetic construct
  misc_feature 1..20: note = synthetic construct; sgRNA
  source 1..20: mol_type = other DNA
caagatctacagcatggggc

SEQ ID NO: 197 - DNA, 20 bp, synthetic construct
  misc_feature 1..20: note = synthetic construct; sgRNA
  source 1..20: mol_type = other DNA
ccctagaaaattgagagaag

SEQ ID NO: 198 - DNA, 20 bp, synthetic construct
  misc_feature 1..20: note = synthetic construct; sgRNA
  source 1..20: mol_type = other DNA
ttgagcagtagtcatgcagg

SEQ ID NO: 199 - DNA, 20 bp, synthetic construct
  misc_feature 1..20: note = synthetic construct; sgRNA
  source 1..20: mol_type = other DNA
gaaagcccaggatgatggga

SEQ ID NO: 200 - DNA, 20 bp, synthetic construct
  misc_feature 1..20: note = synthetic construct; sgRNA
  source 1..20: mol_type = other DNA
ttgattttttgtatgatgtg

SEQ ID NO: 201 - DNA, 20 bp, synthetic construct
  misc_feature 1..20: note = synthetic construct; sgRNA
  source 1..20: mol_type = other DNA
cggggtcgcttgggactctc

SEQ ID NO: 202 - DNA, 20 bp, synthetic construct
  misc_feature 1..20: note = synthetic construct; sgRNA
  source 1..20: mol_type = other DNA
cttcacctctgcacgtcgca

SEQ ID NO: 203 - DNA, 20 bp, synthetic construct
  misc_feature 1..20: note = synthetic construct; sgRNA
  source 1..20: mol_type = other DNA
cctctgcacgtcgcatggag

SEQ ID NO: 204 - DNA, 20 bp, synthetic construct
  misc_feature 1..20: note = synthetic construct; sgRNA
  source 1..20: mol_type = other DNA
aagactgtttgtttaaagac

SEQ ID NO: 205 - DNA, 20 bp, synthetic construct
  misc_feature 1..20: note = synthetic construct; sgRNA
  source 1..20: mol_type = other DNA
gacttctttccttcagtacg

SEQ ID NO: 206 - DNA, 20 bp, synthetic construct
  misc_feature 1..20: note = synthetic construct; sgRNA
  source 1..20: mol_type = other DNA
cggggtcgcttgggactctc

SEQ ID NO: 207 - DNA, 20 bp, synthetic construct
  misc_feature 1..20: note = synthetic construct; sgRNA
  source 1..20: mol_type = other DNA
ccgtctgccgttccgaccga

SEQ ID NO: 208 - DNA, 20 bp, synthetic construct
  misc_feature 1..20: note = synthetic construct; sgRNA
  source 1..20: mol_type = other DNA
gaccgaccacggggcgcacc

SEQ ID NO: 209 - DNA, 20 bp, synthetic construct
  misc_feature 1..20: note = synthetic construct; sgRNA
  source 1..20: mol_type = other DNA
catctgccggaccgtgtgca

SEQ ID NO: 210 - DNA, 20 bp, synthetic construct
  misc_feature 1..20: note = synthetic construct; sgRNA
  source 1..20: mol_type = other DNA
ctgatgacggagagggaata

SEQ ID NO: 211 - DNA, 20 bp, synthetic construct
  misc_feature 1..20: note = synthetic construct; sgRNA
  source 1..20: mol_type = other DNA
gctcctaacccctgggacgc

SEQ ID NO: 212 - DNA, 20 bp, synthetic construct
  misc_feature 1..20: note = synthetic construct; sgRNA
  source 1..20: mol_type = other DNA
atcctggggaagagcacaat

SEQ ID NO: 213 - DNA, 20 bp, synthetic construct
  misc_feature 1..20: note = synthetic construct; sgRNA
  source 1..20: mol_type = other DNA
gcacaatgtccgccccaaaa

SEQ ID NO: 214 - DNA, 20 bp, synthetic construct
  misc_feature 1..20: note = synthetic construct; sgRNA
  source 1..20: mol_type = other DNA
ccgtctgccgttccgaccga

SEQ ID NO: 215 - DNA, 20 bp, synthetic construct
  misc_feature 1..20: note = synthetic construct; sgRNA
  source 1..20: mol_type = other DNA
gaccgaccacggggcgcacc

SEQ ID NO: 216 - DNA, 20 bp, synthetic construct
  misc_feature 1..20: note = synthetic construct; sgRNA
  source 1..20: mol_type = other DNA
catctgccggaccgtgtgca

SEQ ID NO: 217 - DNA, 20 bp, synthetic construct
  misc_feature 1..20: note = synthetic construct; sgRNA
  source 1..20: mol_type = other DNA
cctctgcacgtcgcatggag

SEQ ID NO: 218 - DNA, 20 bp, synthetic construct
  misc_feature 1..20: note = synthetic construct; sgRNA
  source 1..20: mol_type = other DNA
aagactgtttgtttaaagac

SEQ ID NO: 219 - DNA, 20 bp, synthetic construct
  misc_feature 1..20: note = synthetic construct; sgRNA
  source 1..20: mol_type = other DNA
aatattcccagctacaggta

SEQ ID NO: 220 - DNA, 20 bp, synthetic construct
  misc_feature 1..20: note = synthetic construct; sgRNA
  source 1..20: mol_type = other DNA
ctgaagaaaggaagtcatgc

SEQ ID NO: 221 - DNA, 20 bp, synthetic construct
  misc_feature 1..20: note = synthetic construct; sgRNA
  source 1..20: mol_type = other DNA
aaggaagtcatgctctagaa

SEQ ID NO: 222 - DNA, 20 bp, synthetic construct
  misc_feature 1..20: note = synthetic construct; sgRNA
  source 1..20: mol_type = other DNA
aagtcatgctctagaagatc

SEQ ID NO: 223 - DNA, 20 bp, synthetic construct
  misc_feature 1..20: note = synthetic construct; sgRNA
  source 1..20: mol_type = other DNA
catgctctagaagatctatg

SEQ ID NO: 224 - DNA, 20 bp, synthetic construct
  misc_feature 1..20: note = synthetic construct; sgRNA
  source 1..20: mol_type = other DNA
tctatggcggagtcgagaca